(19) United States
(12) Patent Application Publication
(10) Pub. No.: US 2023/0238124 A1
(43) Pub. Date: Jul. 27, 2023

(54) REMOTE CONFIGURATION OF A RESPIRATORY DEVICE

(71) Applicant: ResMed Pty Ltd, Bella Vista, NSW (AU)

(72) Inventors: Lisa Nicole Matthews, Como (AU); Millen James Rath-May, Sydney (AU); My Hong Vuong Salameh, Bella Vista, NSW (AU)

(21) Appl. No.: 18/002,549

(22) PCT Filed: Jun. 25, 2021
(86) PCT No.: PCT/AU2021/050665

§ 371 Date: Dec. 20, 2022

(30) Foreign Application Priority Data
Jun. 26, 2020 (AU) 2020902141

(51) Int. Cl. G16H 40/40; G16H 40/67

(52) U.S. Cl. CPC G16H 40/40; G16H 40/67

(57) ABSTRACT

The present technology relates to systems and/or methods for enabling a respiratory device to be configured when a clinician or healthcare professional is remote from the respiratory device. One form provides a method of configuring a respiratory device, the respiratory device comprising a processor configured to control operation of the respiratory device in accordance with a plurality of operating parameters. The method comprises determining a combination of settings for the device from an identifier sent to the device, the identifier corresponding to the combination of settings, and configuring the respiratory device accordingly. Another form provides a method of verifying the configuration of the respiratory device by outputting an identifier corresponding to the combination of settings for the device, and determining the settings from the identifier.

### 1 CROSS REFERENCE TO RELATED APPLICATIONS

This application claims the benefit of Australian Patent Application No. 2020902141 filed 26 Jun. 2020, the contents of which are incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

The present technology relates generally to systems and methods for screening, diagnosis, monitoring, amelioration, treatment or prevention of a respiratory disorder; more particularly the present technology relates to systems and/or methods for enabling a respiratory device to be configured when a clinician or healthcare professional is remote from the respiratory device.

### 2.2 Description of the Related Art

A range of respiratory disorders exist. Certain disorders may be characterised by particular events, e.g. apneas, hypopneas, and hyperpneas. Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders. Respiratory pressure therapy is the application of a supply of air to an entrance to the airways at a controlled target pressure that is nominally positive with respect to atmosphere throughout the patient’s breathing cycle (in contrast to negative pressure therapies such as the tank ventilator or cuirass). CPAP, NIV and IV are examples of respiratory pressure therapy.

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient’s airways is ‘open’ (unsealed) and the respiratory therapy may only supplement the patient’s own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient’s peak inspiratory flow rate. HFT has been used to treat OSA, CSR, respiratory failure, COPD, and other respiratory disorders. As an alternative to constant flow rate, the treatment flow rate may follow a profile that varies over the respiratory cycle.

Another form of flow therapy is long-term oxygen therapy (LTOT) or supplemental oxygen therapy. For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

Another form of respiratory therapy is oxygen concentration. An oxygen concentrator is a device that concentrates the amount of oxygen in a gas supply to provide an oxygen-enriched flow of breathable gas to a patient. Some forms of oxygen concentrators operate by taking ambient air and selectively reducing its nitrogen content to produce the oxygen-enriched flow of breathable gas.

Another form of respiratory therapy is ventilation. A ventilator is a device that causes breathable air to move into and/or out of the lungs to enable a patient to breathe where the patient is unable to breathe themselves, or requires assistance to do so. A ventilator creates the flow of air through a mechanical mechanism.

These respiratory therapies may be provided by a respiratory therapy system or device. Such systems and devices may also be used to screen, diagnose, or monitor a condition without treating it. A respiratory therapy system may comprise a Respiratory Pressure Therapy Device (RPT device), an air circuit, a humidifier, a patient interface, an oxygen source, and data management.

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient’s face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. The flow of air may be pressurised. Examples of RPT devices include a CPAP device, NIV device, HFT device, oxygen concentrator and a ventilator.

Delivery of a flow of air without humidification may cause drying of airways. The use of a humidifier with an RPT device and the patient interface produces humidified gas that minimizes drying of the nasal mucosa and increases patient airway comfort. In addition in cooler climates, warm air applied generally to the face area in and about the patient interface is more comfortable than cold air. Humidifiers therefore often have the capacity to heat the flow of air as well as humidifying it.

RPT devices are typically configurable by altering a number of operating parameters to particular settings for each use and/or each patient. Many of the operating parameters in an RPT device are therapy-related, for example the pressure of the flow of air in CPAP therapy and the treatment flow rate in HFT. Other operating parameters serve other purposes, for example they help the therapy be more comfortable for a patient, for example by controlling humidification of the flow of air. Other operating parameters relate to the usability of the RPT device, for example user interface settings.

Settings for RPT device operating parameters, particularly therapy parameters, are typically determined by, or in consultation with, a clinician. These settings may be important in ensuring that the patient receives the respiratory therapy they need. It is therefore important that the RPT device is correctly configured to deliver respiratory therapy according to the appropriate operating settings.

Configuration of an RPT device may occur when a patient first commences respiratory therapy. In addition, a patient’s condition may change over the course of a period receiving respiratory therapy, for example as a result of the therapy. Consequently, a clinician may diagnose a change to the respiratory therapy, and this may necessitate a re-configuration of the settings of the RPT device. A patient’s response to therapy may also create a need to re-configure the RPT device’s settings, for example if the patient’s condition does not ameliorate or if it ameliorates at a different rate to what is expected.

Many RPT devices are used by a patient at home without a clinician being present. Often a clinician will help a patient initially configure their home RPT device with the appropriate operating settings to deliver the required respiratory therapy. This may be time-consuming as it requires the clinician to visit the patient’s home. This may also be costly since demands on a clinician’s time adds cost to the health system within which they work. In some cases the patient may be so inaccessible that a home visit by a clinician is impractical.

To address these problems clinicians sometimes provide instructions to patients to assist the patients in configuring the RPT devices themselves. However this can result in patients not configuring the RPT devices correctly through human error resulting from a lack of understanding or lack of familiarity with the RPT device, or poor instructions.

Some RPT devices are used in a hospital or other healthcare facility. In a hospital environment clinicians and other healthcare professionals are more likely to be physically present to ensure appropriate configuration of the RPT device. Nevertheless, configuration errors may still occur as the result of human error. In addition, periods of high demand on a healthcare system create more demands on clinicians’ time and may make it more difficult for a clinician to dedicate enough time to all patients to ensure appropriate configuration of RPT devices.

The use of an incorrectly configured RPT device may result in a patient receiving sub-optimal, ineffective or potentially harmful respiratory therapy.

Patients receiving respiratory therapy may be assessed by their respiratory clinician after commencing respiratory therapy. Such assessments often involve a check of the RPT device settings being used by the patient. This check may involve the clinician looking at each operating setting of the RPT device in turn, and may be a time-consuming exercise. It may be particularly time-consuming where the respiratory therapy is provided at a patient’s home and a clinician needs to visit the patient’s home in order to conduct the check.

There is a need for a respiratory device, a respiratory system, a method of configuring a respiratory device and/or a method of verifying the configuration of a respiratory device that addresses any one or more of these problems.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of the present technology relates to systems and/or methods for enabling a user to configure a respiratory device in a quick, convenient and/or low-error manner. One form of the technology relates to systems and/or methods for enabling a respiratory device to be configured when a clinician or healthcare professional is remote from the respiratory device.

One form of the present technology comprises a respiratory device. The respiratory device includes at least one memory having processor-readable instructions, and at least one processor for executing the processor-readable instructions. The processor-readable instructions include instructions for causing the processor, upon receipt of an identifier, to determine from the identifier a combination of settings for the respiratory device, and to cause the respiratory device to operate in accordance with the determined combination of settings.

Another form of the present technology comprises a system for use in configuring a respiratory device. The system includes at least one memory having processor-readable instructions, and at least one processor for executing the processor-readable instructions. The processor-readable instructions include instructions for causing the processor, upon receipt of a combination of settings for the respiratory device, to generate from the combination of settings an identifier corresponding to the combination of settings.

One form of the present technology comprises a processor-implemented method of configuring a respiratory device. The respiratory device may comprise a processor configured to control operation of the respiratory device in accordance with a plurality of operating parameters, each of the plurality of operating parameters being able to be set to a plurality of settings. The method may comprising receiving an identifier, wherein the identifier is one identifier out of a plurality of identifiers, wherein each of the plurality of identifiers corresponds to a combination of settings out of a plurality of combinations of settings, wherein each of the combinations of settings comprises a setting out of the plurality of settings for each of the plurality of operating parameters. The method may further comprise determining, from the received identifier, the combination of settings corresponding to the received identifier. The method may further comprise configuring the respiratory device to operate in accordance with the determined combination of settings.

In one example of this form of the technology, the combination of settings corresponding to each of the plurality of identifiers is unique to each identifier.

In one example of this form of the technology, the identifier is received as data representative of a character string.

In one example of this form of the technology, the method comprises receiving the identifier from a user input device. The user input device may be a mobile computing device configured to send the identifier directly or indirectly to the processor.

In one example of this form of the technology, the step of receiving the identifier comprises receiving optical data representative of an optically machine-readable code, and generating the identifier from the optical data.

In one example of this form of the technology, the step of receiving the identifier comprises receiving acoustic data representative of a plurality acoustic tones, and generating the identifier from the acoustic data.

In one example of this form of the technology, the user input device is a keypad on the respiratory device.

In one example of this form of the technology, the step of determining the combination of settings from the received identifier may comprise identifying the combination of settings corresponding to the received identifier in a data array, the data array storing each of the plurality of identifiers and the corresponding combination of settings out of the plurality of combination of settings in relation to each other.

In one example of this form of the technology, the method may further comprise validating the received identifier.

In one example of this form of the technology, the step of validating the received identifier comprises validating the received identifier as corresponding to an intended combination of settings.

In one example of this form of the technology, the step of validating the received identifier may comprise receiving first validation data, the first validation data generated from the received identifier using a validation data generation algorithm. The step of validating may further comprise comparing the first validation data and the received identifier to validate the received identifier.

In one example of this form of the technology, the step of comparing may comprise generating second validation data from the received identifier using the validation data generation algorithm. The step of comparing may further comprise comparing the first validation data with the second validation data.

In one example of this form of the technology, the step of comparing may comprise generating a validation identifier from the first validation data using a validation identifier generation algorithm, the validation identifier generation algorithm being inverse to the validation data generation algorithm. The step of comparing may further comprise comparing the validation identifier with the received identifier.

In one example of this form of the technology, the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

Another form of the present technology comprises a processor-implemented method of generating an identifier for use in configuring a respiratory device. The method may comprise receiving a combination of settings, wherein the combination of settings is one combination of settings out of a plurality of combinations of settings, and wherein each of the combinations of settings comprises a setting out of a plurality of settings for each of a plurality of operating parameters of the respiratory device. The method may further comprise determining, from the received combination of settings, an identifier, wherein the identifier is one identifier out of a plurality of identifiers, wherein each of the plurality of identifiers corresponds to one combination of settings out of the plurality of combinations of settings. The method may further comprise outputting the identifier.

In one example of this form of the technology, the combination of settings corresponding to each of the plurality of identifiers is unique to each identifier.

In one example of this form of the technology, the identifier is output as data representative of a character string.

In one example of this form of the technology, the identifier is sent to a mobile computing device.

In one example of this form of the technology, the identifier is output as optical data representative of an optically machine-readable code.

In one example of this form of the technology, the identifier is output as acoustic data representative of a plurality acoustic tones.

In one example of this form of the technology, the step of determining the identifier from the received combination of settings comprises identifying the identifier corresponding to the received combination of settings in a data array, the data array storing each of the plurality of identifiers and the corresponding combination of settings out of the plurality of combination of settings in relation to each other.

In one example of this form of the technology, the method further comprises generating validation data to enable validation of the identifier.

In one example of this form of the technology, the step of generating validation data comprises applying a validation data generation algorithm to the identifier to generate the validation data.

In one example of this form of the technology, the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

Another aspect of the present technology relates to systems and/or methods for enabling a user to verify the configuration of a respiratory device in a quick, convenient and/or low-error manner.

One form of the present technology comprises a respiratory device. The respiratory device includes at least one memory having processor-readable instructions, and at least one processor for executing the processor-readable instructions. The processor-readable instructions include instructions for causing the processor to generate an identifier corresponding to a combination of present settings of the respiratory device.

Another form of the present technology comprises a system for verifying the configuration of a respiratory device. The system includes at least one memory having processor-readable instructions, and at least one processor for executing the processor-readable instructions. The processor-readable instructions include instructions for causing the processor, upon receipt of an identifier, to determine from the identifier a combination of settings for the respiratory device, and to output the combination of settings for verification by a user.

One form of the present technology comprises a processor-implemented method of generating an identifier for use in verifying the configuration of a respiratory device, the respiratory device comprising a processor configured to control operation of the respiratory device in accordance with a plurality of operating parameters, each of the plurality of operating parameters being able to be set to a plurality of settings. The method may comprise receiving a present combination of settings of the respiratory device, wherein the present combination of settings is one combination of settings out of a plurality of combinations of settings for the respiratory device, and wherein each of the combinations of settings comprises a setting out of the plurality of settings for each of the plurality of operating parameters of the respiratory device. The method may further comprise determining, from the received combination of settings, an identifier, wherein the identifier is one identifier out of a plurality of identifiers, wherein each of the plurality of identifiers corresponds to one combination of settings out of the plurality of combinations of settings. The method may further comprise outputting the identifier.

In one example of this form of the technology, the combination of settings corresponding to each of the plurality of identifiers is unique to each identifier.

In one example of this form of the technology, the identifier is output as data representative of a character string.

In one example of this form of the technology, the identifier is output to a display of the respiratory device.

In one example of this form of the technology, the identifier is output as optical data representative of an optically machine-readable code.

In one example of this form of the technology, the identifier is output as acoustic data representative of a plurality acoustic tones.

In one example of this form of the technology, the step of determining the identifier from the received combination of settings comprises identifying the identifier corresponding to the received combination of settings in a data array, the data array storing each of the plurality of identifiers and the corresponding combination of settings out of the plurality of combination of settings in relation to each other.

In one example of this form of the technology, the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

Another form of the present technology comprises a processor-implemented method of verifying the configuration of a respiratory device. The method may comprise receiving an identifier, wherein the identifier is one identifier out of a plurality of identifiers, wherein each of the plurality of identifiers corresponds to a present combination of settings out of a plurality of combinations of settings, wherein each of the combinations of settings comprises a setting out of the plurality of settings for each of the plurality of operating parameters. The method may further comprise determining, from the received identifier, the present combination of settings corresponding to the received identifier. The method may further comprise outputting the present combination of settings.

In one example of this form of the technology, the combination of settings corresponding to each of the plurality of identifiers is unique to each identifier.

In one example of this form of the technology, the identifier is received as data representative of a character string.

In one example of this form of the technology, the method comprises receiving the identifier from a user input device.

In one example of this form of the technology, the user input device is a mobile computing device configured to send the identifier directly or indirectly to the processor.

In one example of this form of the technology, the step of receiving the identifier comprises receiving optical data representative of an optically machine-readable code, and generating the identifier from the optical data.

In one example of this form of the technology, the step of receiving the identifier comprises receiving acoustic data representative of a plurality acoustic tones, and generating the identifier from the acoustic data.

In one example of this form of the technology, the step of determining the present combination of settings from the received identifier comprises identifying the combination of settings corresponding to the received identifier in a data array, the data array storing each of the plurality of identifiers and the corresponding combination of settings out of the plurality of combination of settings in relation to each other.

In one example of this form of the technology, the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

One form of the present technology comprises a respiratory device. The respiratory device may comprise a flow generator for generating a flow of air for delivery to a patient’s airways. The respiratory device may further comprise a processor configured to perform the processor-implemented method of any one or more other forms of the present technology.

One form of the present technology comprises a system. The system may comprise a processor configured to perform the processor-implemented method of another form of the present technology.

An aspect of certain forms of the present technology is a medical device that is easy to use, e.g. by a person who does not have medical training, by a person who has limited dexterity, vision or by a person with limited experience in using this type of medical device.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

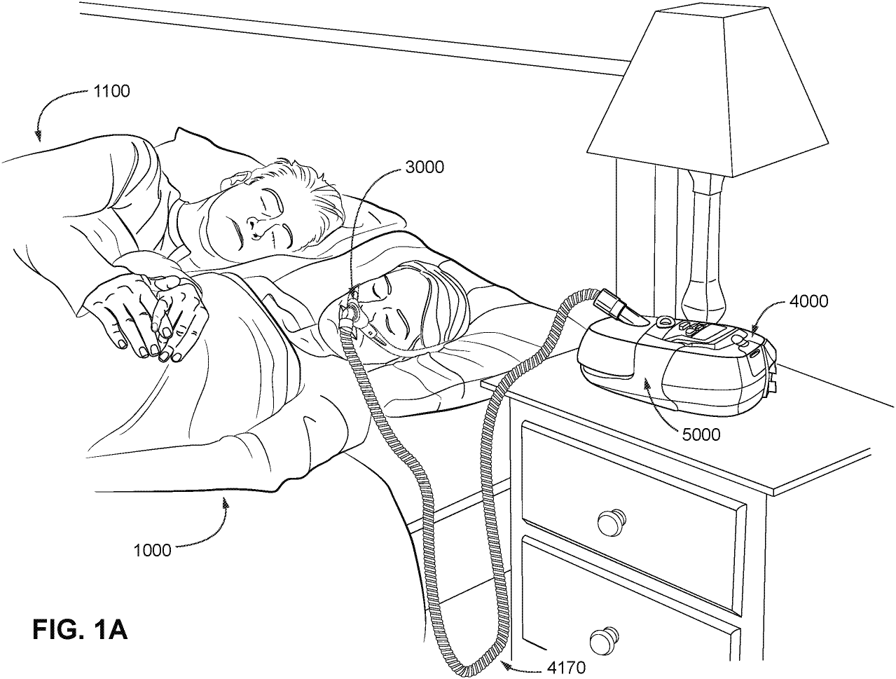

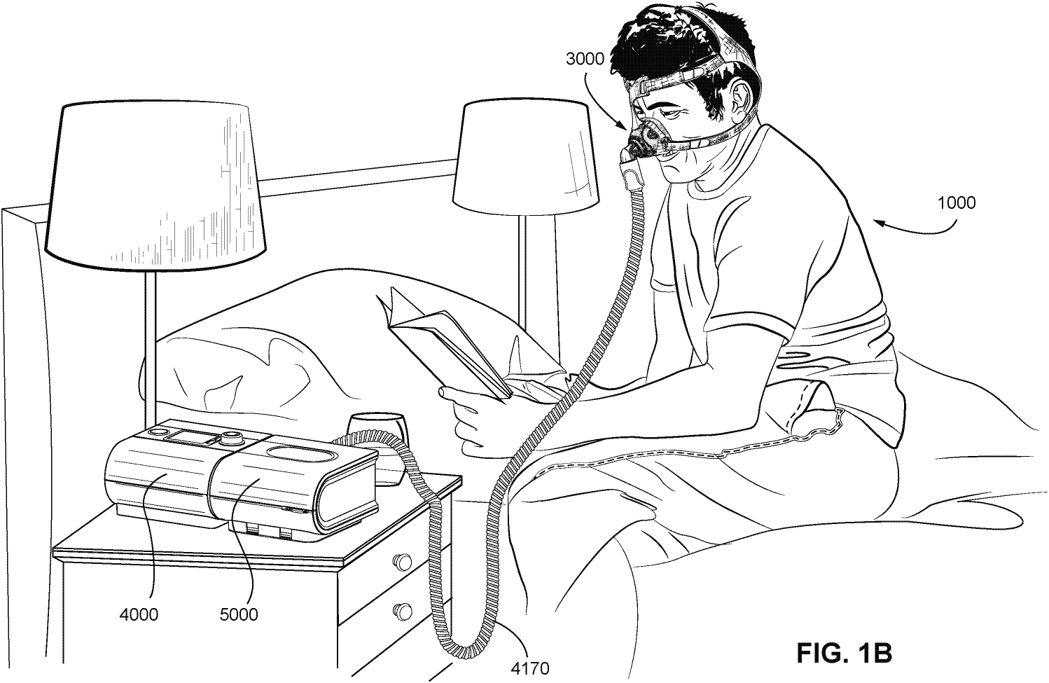

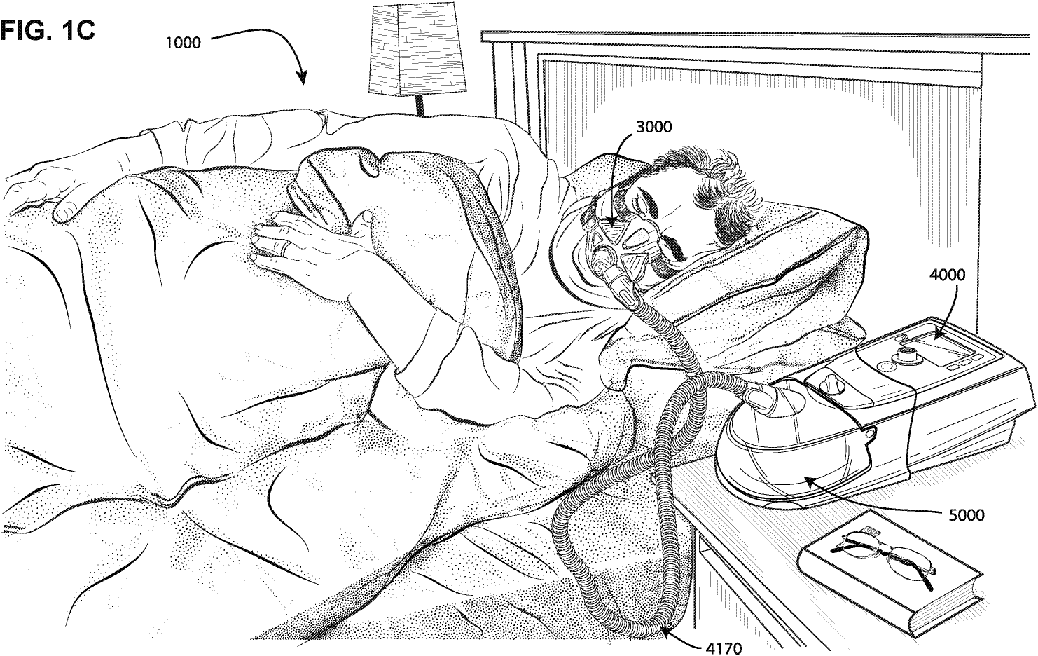

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

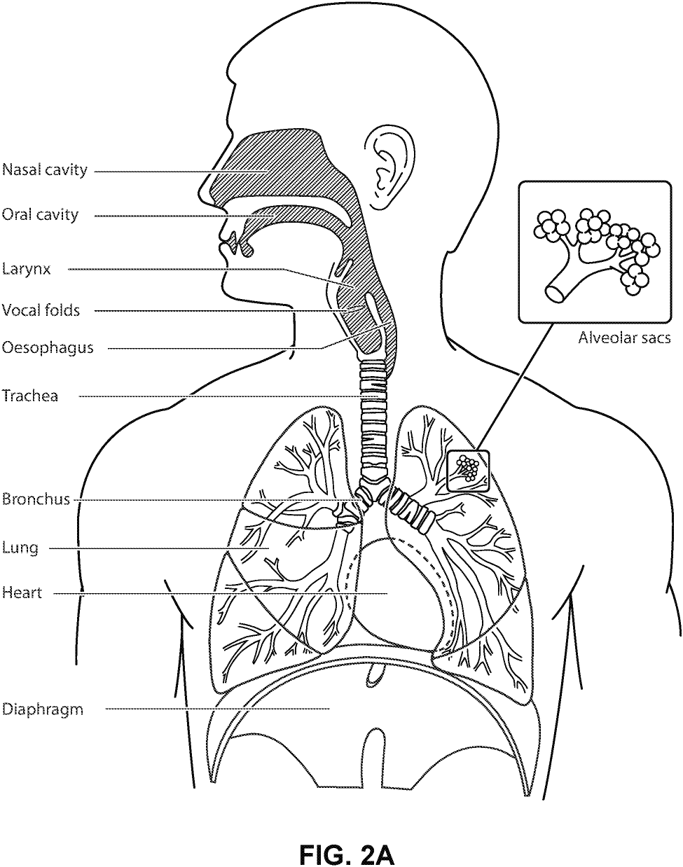

FIG. 2A shows an overview of a human respiratory system including the nasal and oral cavities, the larynx, vocal folds, oesophagus, trachea, bronchus, lung, alveolar sacs, heart and diaphragm.

### 4.3 Patient Interface

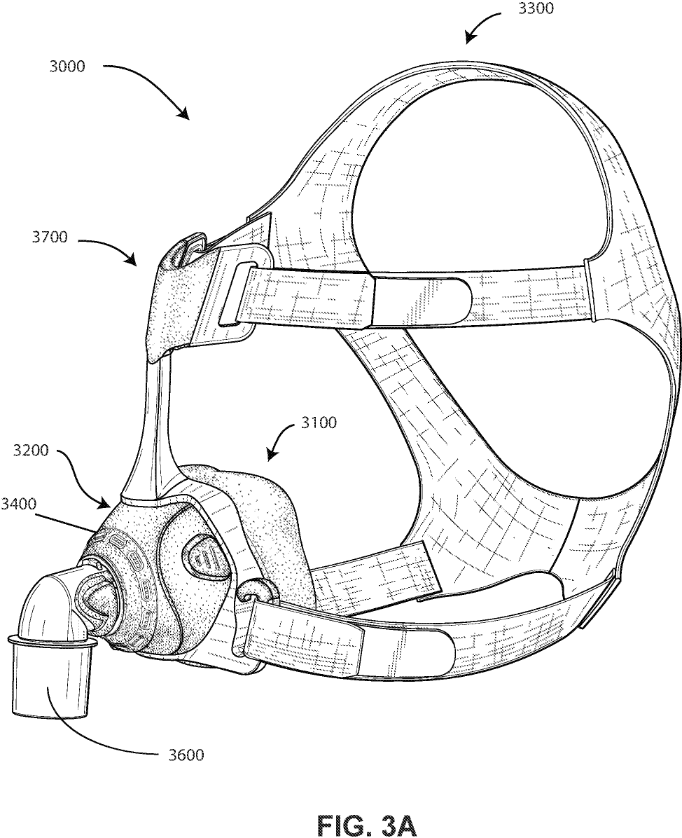

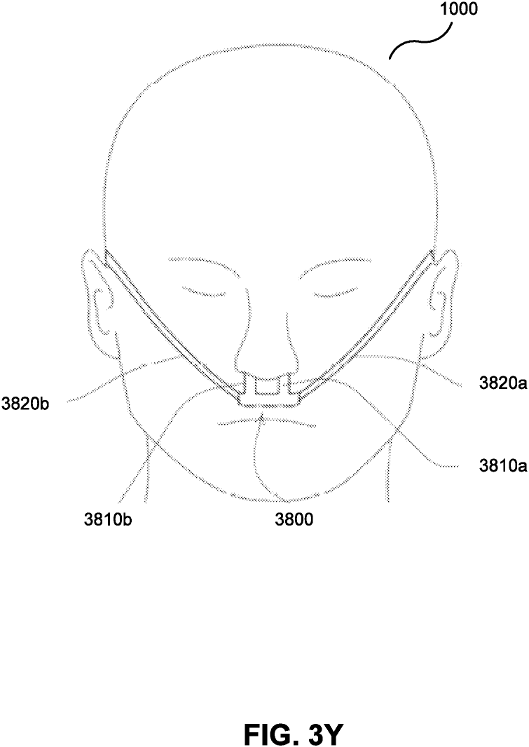

FIG. 3A shows a patient interface in the form of a nasal mask in accordance with one form of the present technology.

FIG. 3B shows a patient interface in the form of a nasal cannula in accordance with one form of the present technology.

### 4.4 Rpt Device

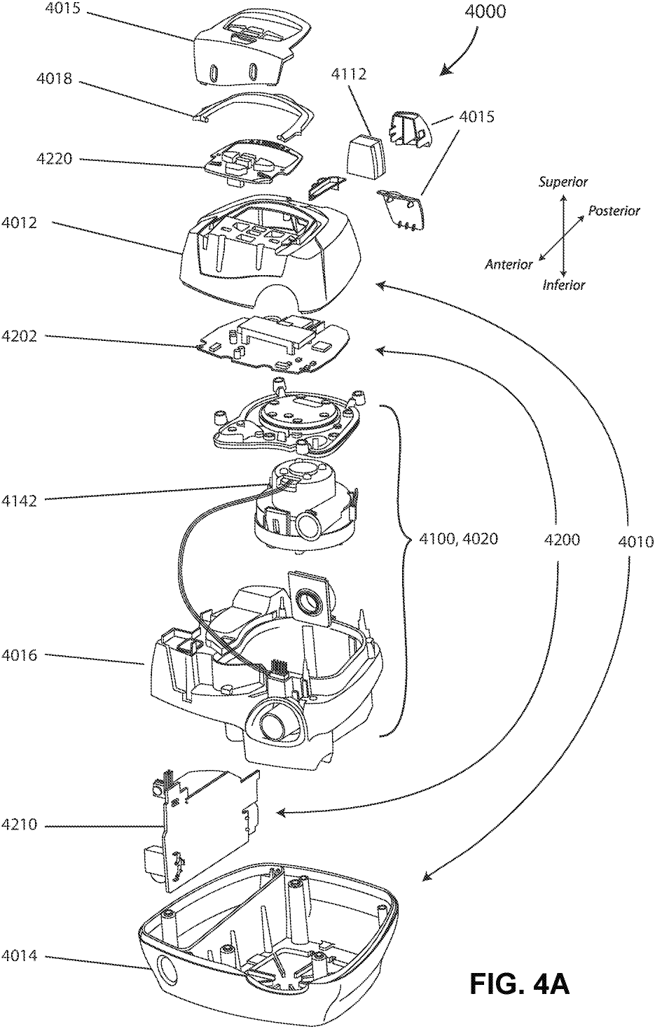

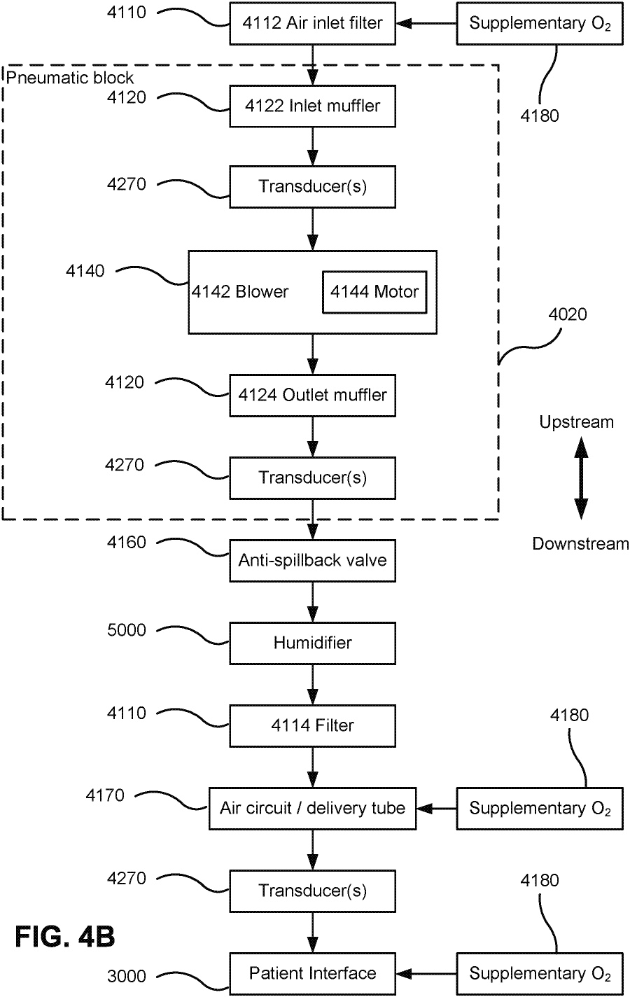

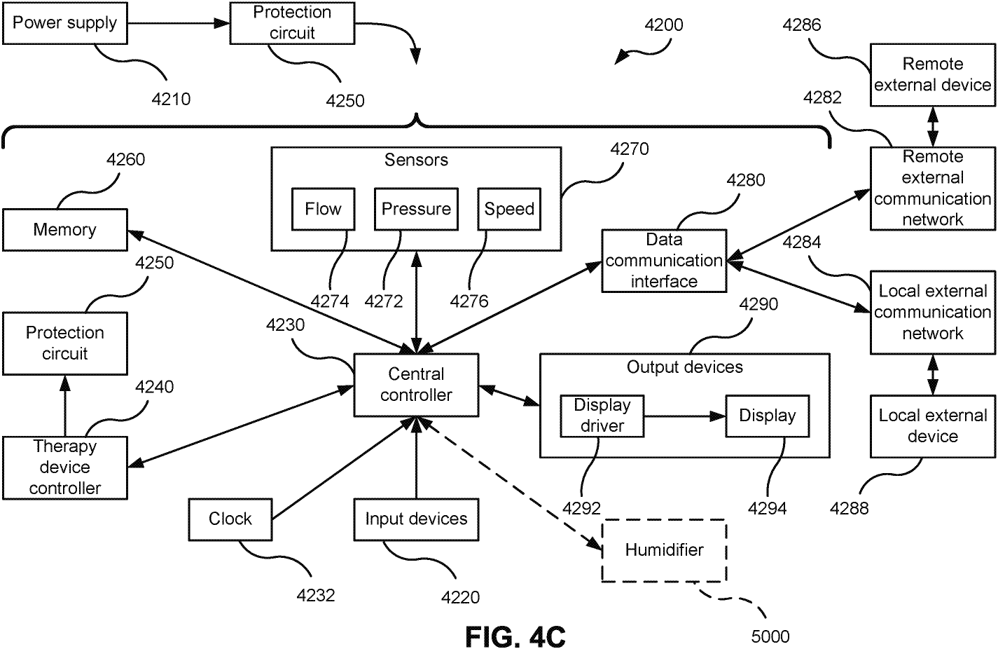

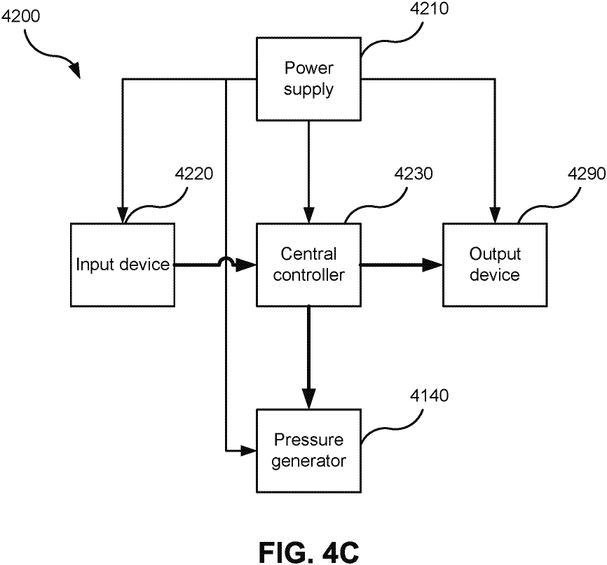

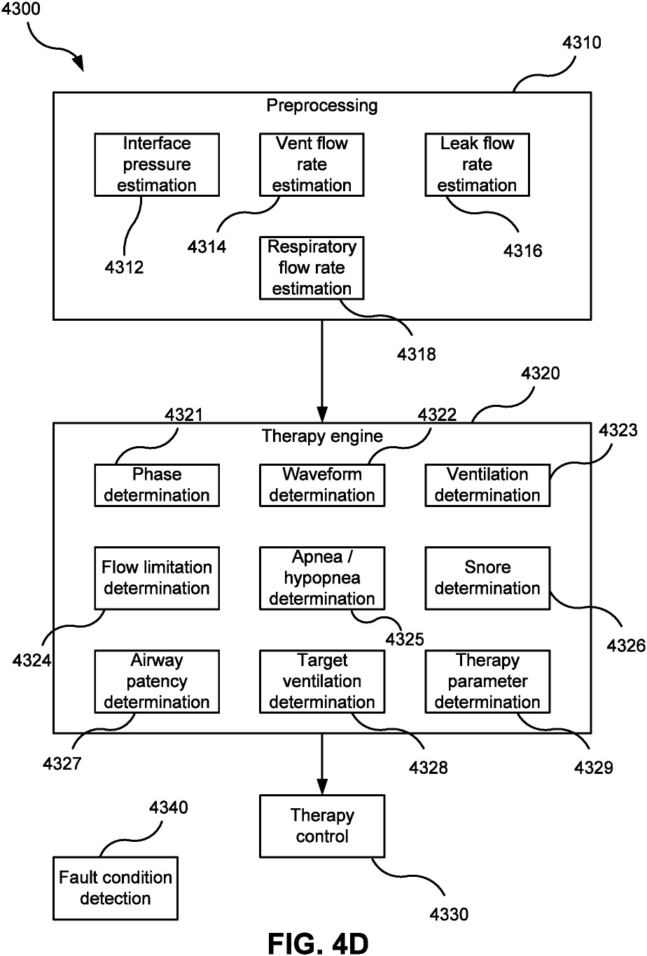

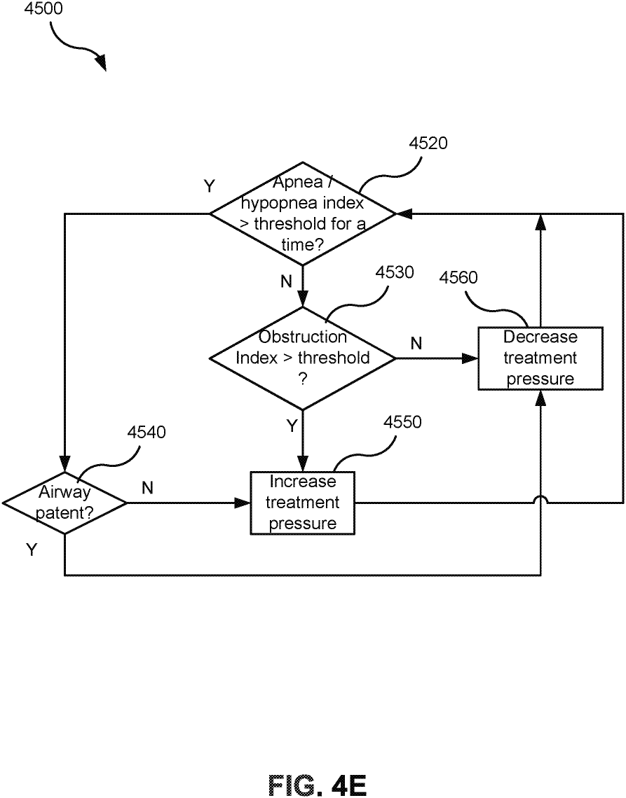

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a further schematic diagram of the electrical components of an RPT device in accordance with one form of the present technology.

FIG. 4E is a schematic diagram of the algorithms implemented in an RPT device in accordance with one form of the present technology.

FIG. 4F is a flow chart illustrating a method carried out by the therapy engine module of FIG. 4E in accordance with one form of the present technology.

### 4.5 Humidifier

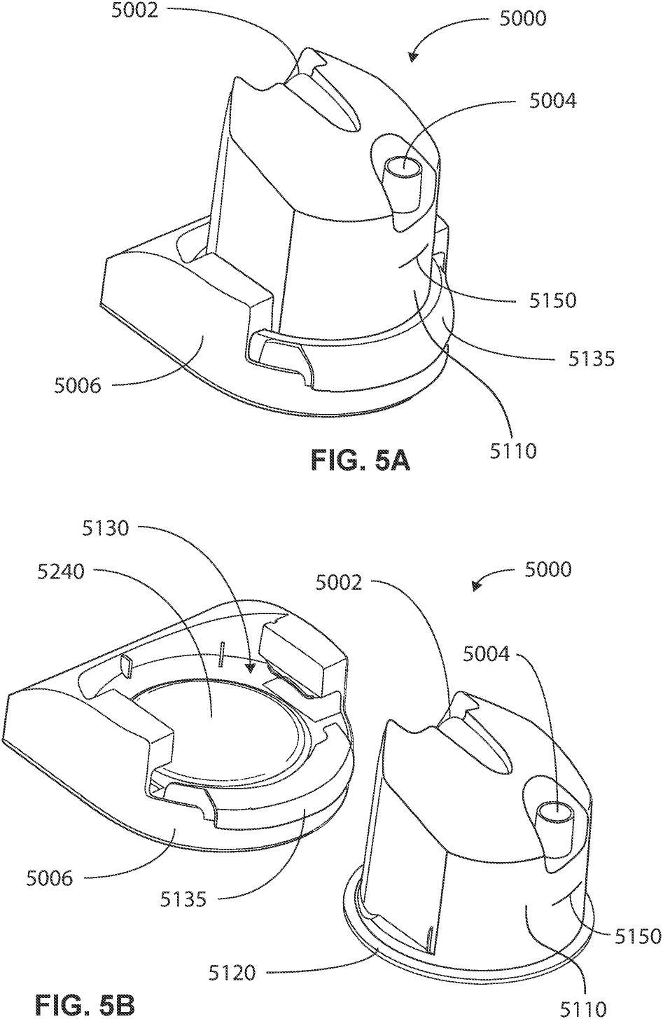

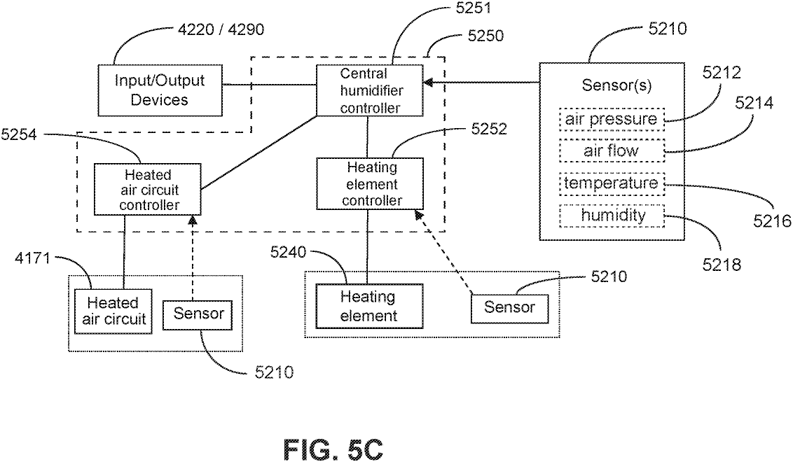

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

### 4.6 Breathing Waveforms

FIG. 6A shows a model typical breath waveform of a person while sleeping.

### 4.7 Screening, Diagnosis and Monitoring Systems

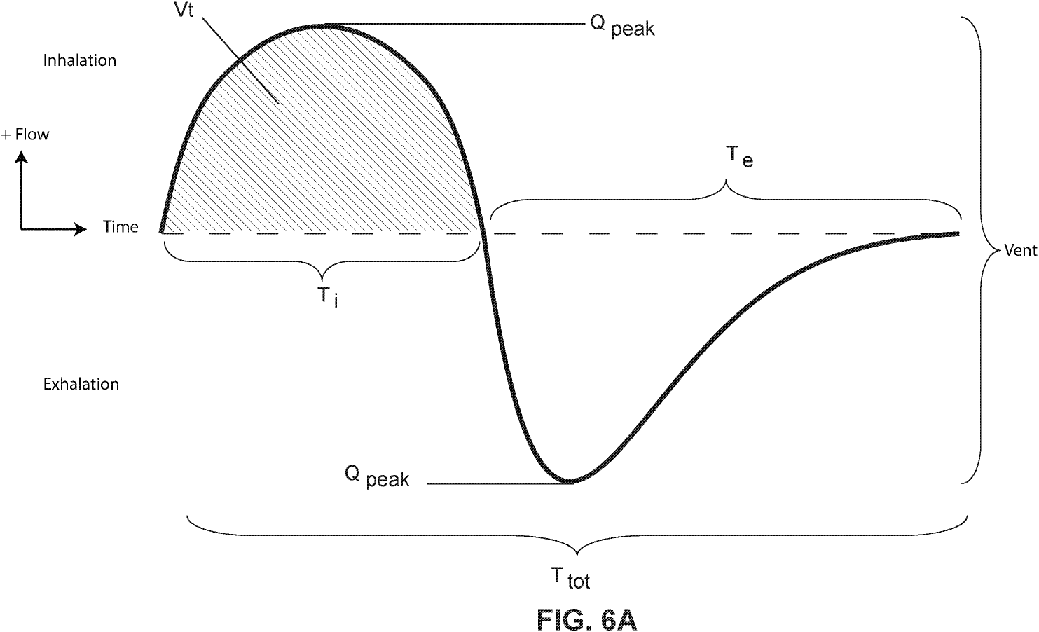

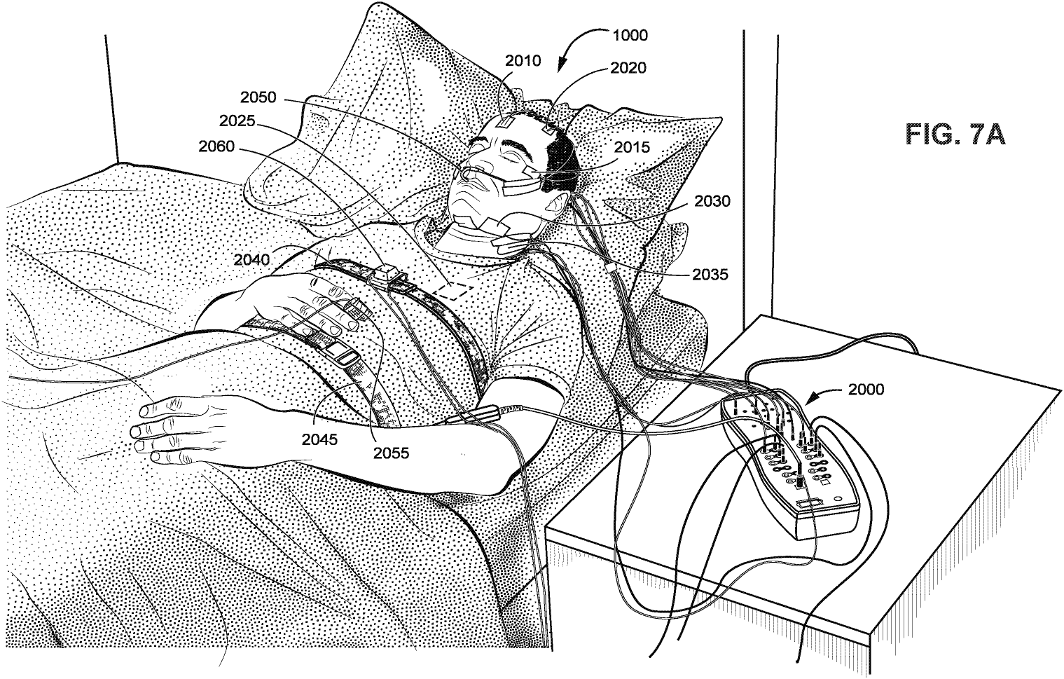

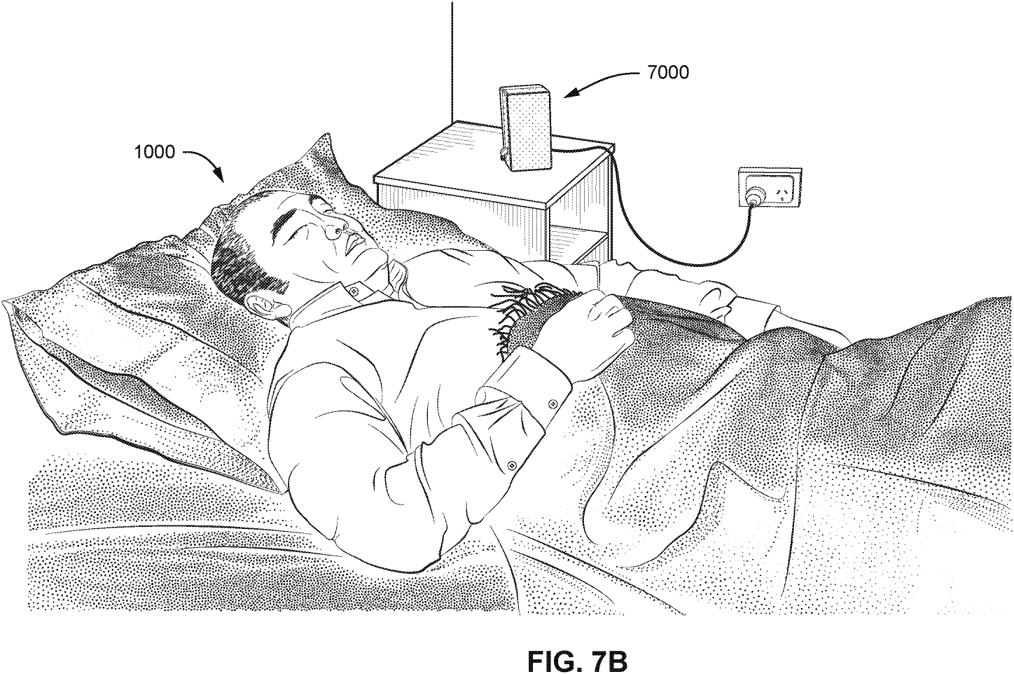

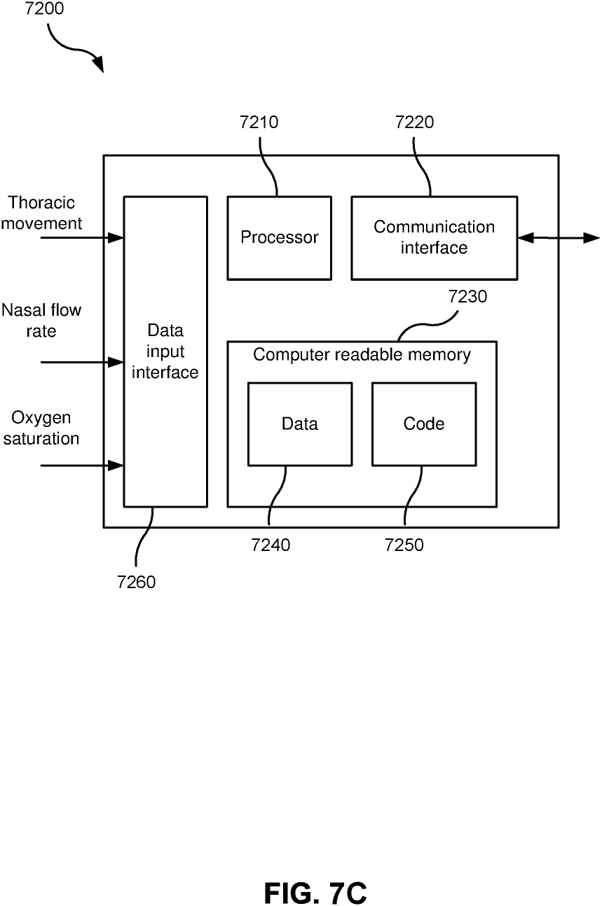

FIG. 7A shows a patient undergoing polysomnography (PSG). The patient is sleeping in a supine sleeping position.

FIG. 7B shows a monitoring apparatus for monitoring the condition of a patient. The patient is sleeping in a supine sleeping position.

FIG. 7C is a schematic diagram of the components of a screening / diagnosis / monitoring device that may be used to implement an Respiratory polygraphy (RPG) headbox in an RPG screening / diagnosis / monitoring system concentrator in accordance with one form of the present technology.

### 4.8 Oxygen Concentrator

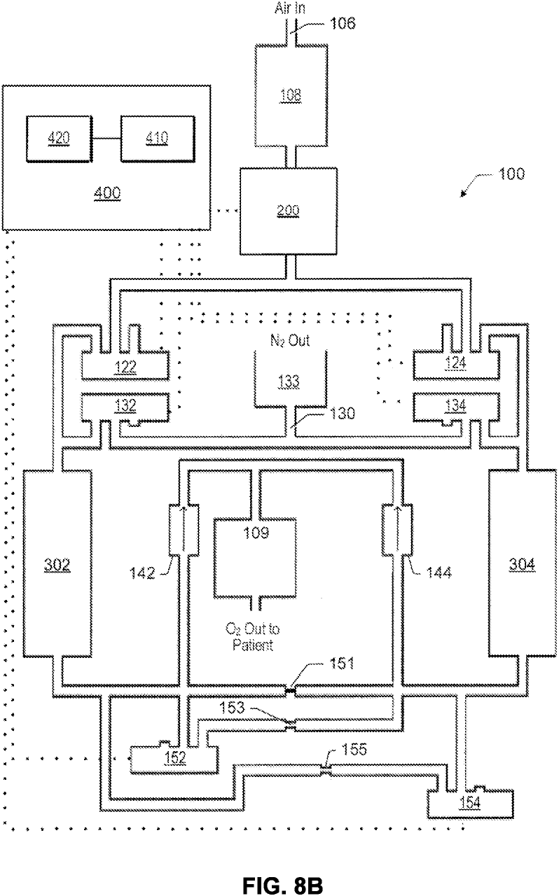

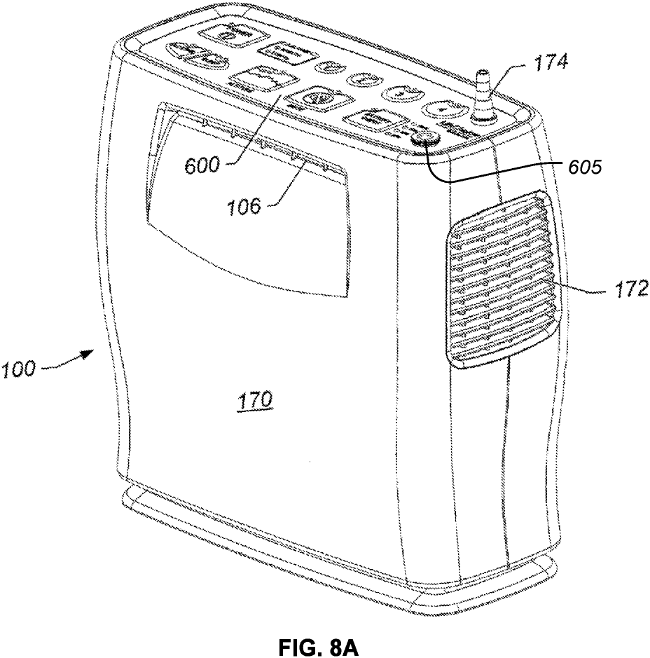

FIG. 8A depicts an oxygen concentrator in accordance with one form of the present technology.

FIG. 8B is a schematic diagram of the components of the oxygen concentrator of FIG. 8A.

### 4.9 Computing System and Processes

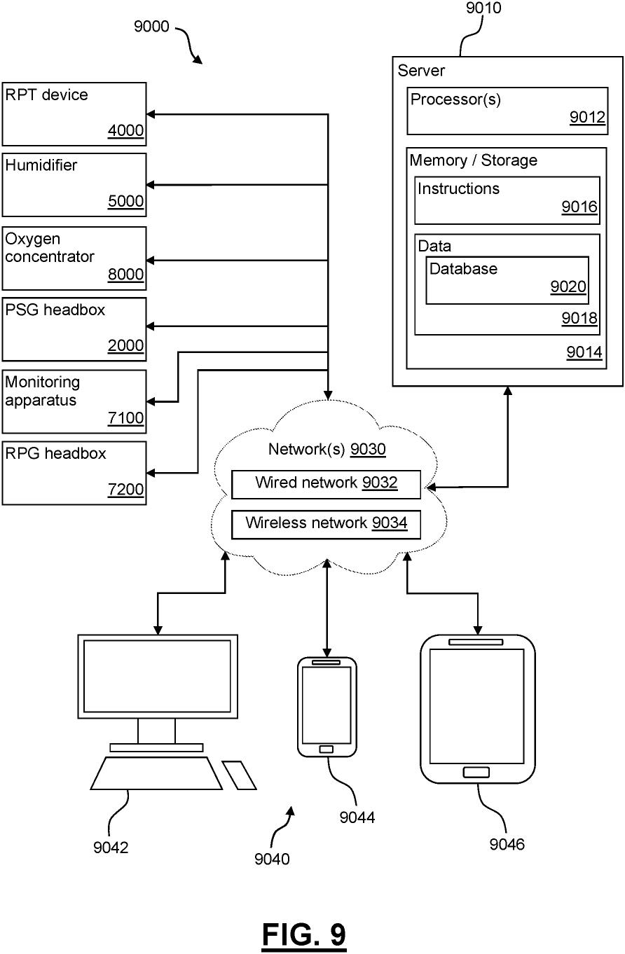

FIG. 9 is a diagram of an example system for implementing methods according to forms of the technology, the system including a computing device.

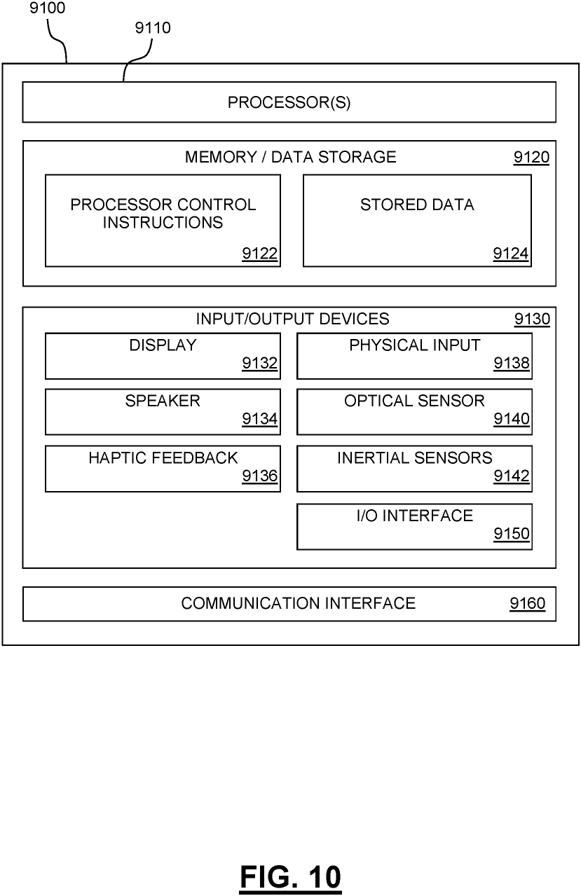

FIG. 10 is a diagram of the components of an example computing device used to implement methods according to forms of the technology.

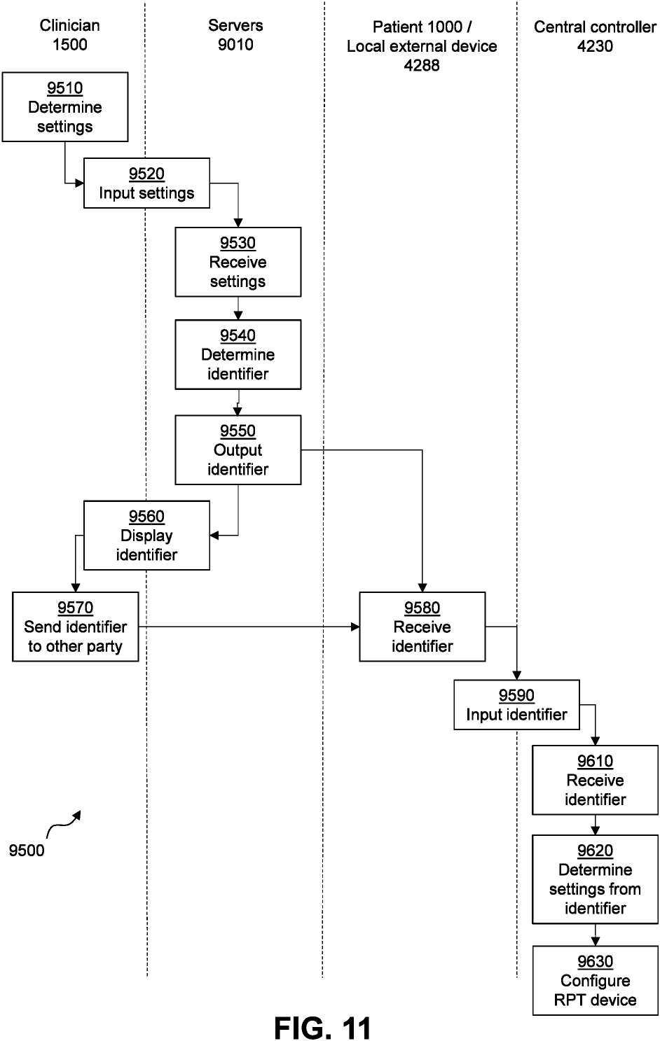

FIG. 11 is flow diagram of a method of configuring an RPT device according to certain forms of the technology.

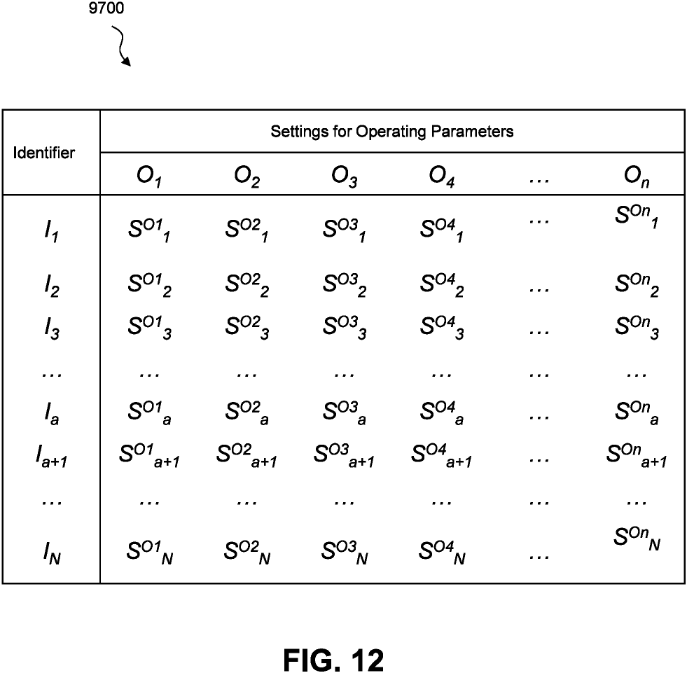

FIG. 12 is a diagram of a data array for performing an identifier determination algorithm and/or a combination of settings determination algorithm according to forms of the technology.

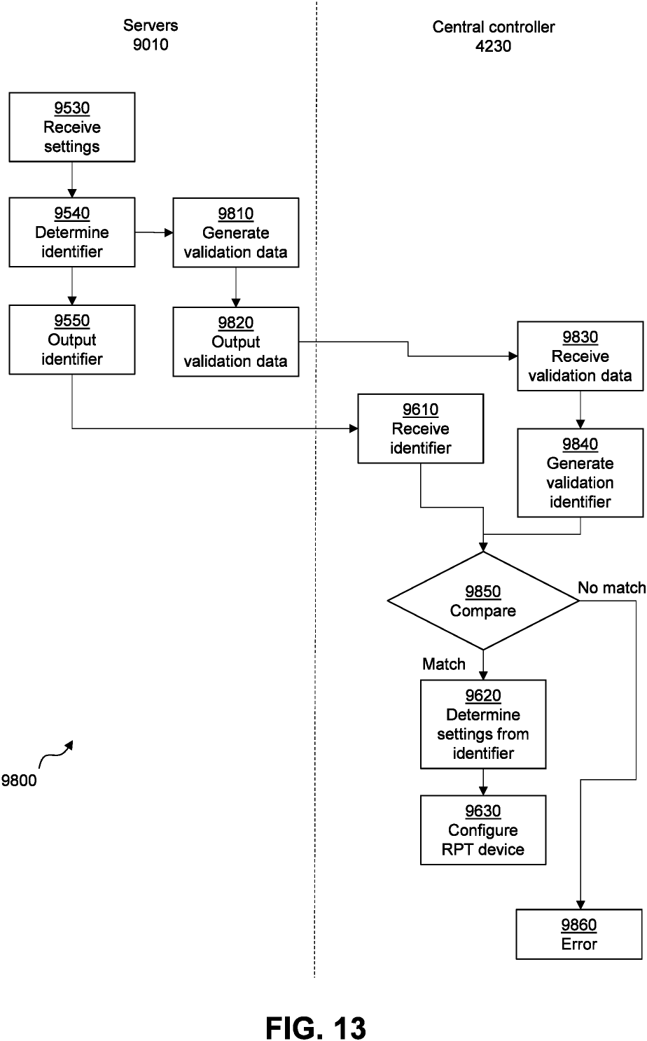

FIG. 13 is a flow diagram of a method of validating an identifier according to one form of the technology.

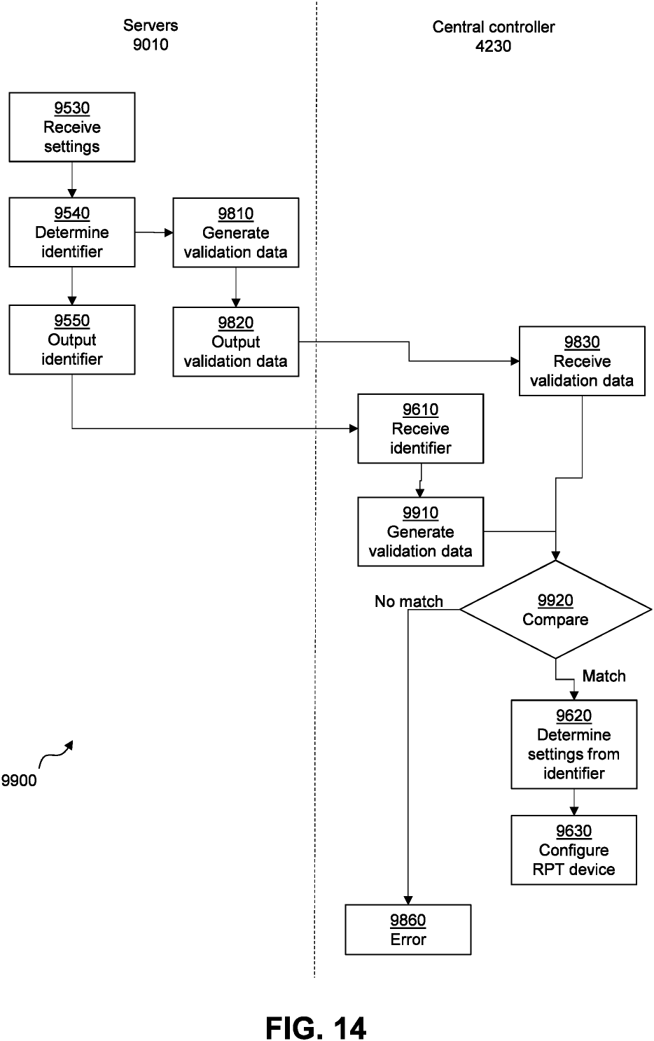

FIG. 14 is a flow diagram of a method of validating an identifier according to another form of the technology.

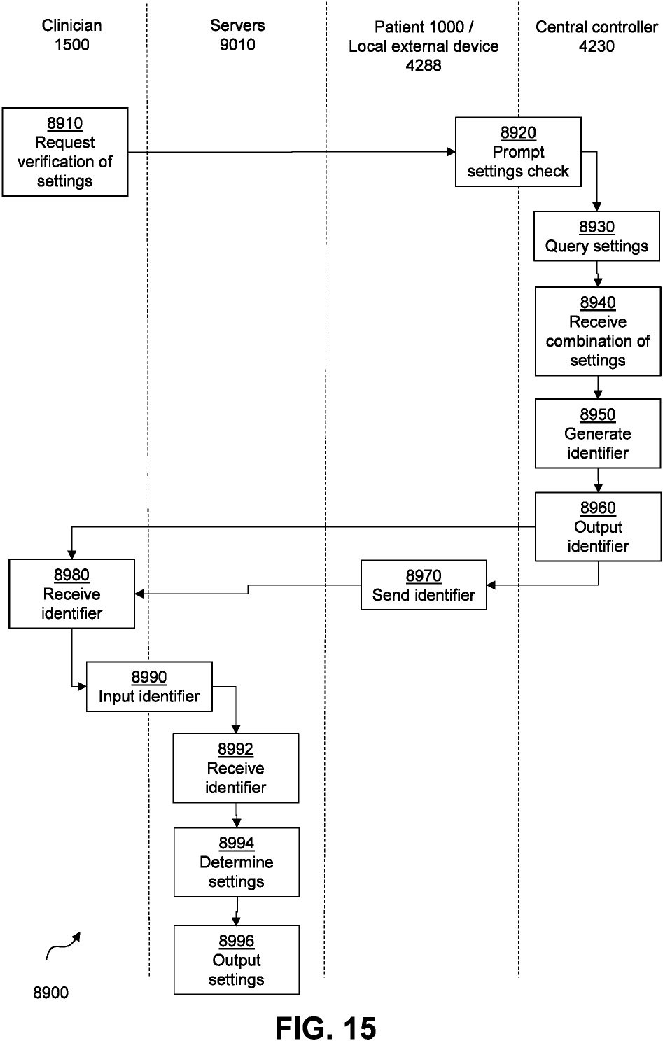

FIG. 15 is a flow diagram of a method of verifying the configuration RPT device according to certain forms of the technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may be suitable for delivering any type of respiratory therapy including, but not limited to, continuous positive airway pressure (CPAP) therapy, non-invasive ventilation (NIV), invasive ventilation (IV), high flow therapy (HFT), oxygen concentration and ventilation.

The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and optionally a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

In accordance with another form of the technology there is provided an unsealed patient interface 3800, in the form of a nasal cannula, which includes nasal prongs 3810a, 3810b which can deliver air to respective nares of the patient 1000 via respective orifices in their tips. Such nasal prongs do not generally form a seal with the inner or outer skin surface of the nares. The air to the nasal prongs may be delivered by one or more air supply lumens 3820a, 3820b that are coupled with the nasal cannula 3800. The lumens 3820a, 3820b lead from the nasal cannula 3800 to a respiratory therapy device via an air circuit. The unsealed patient interface 3800 is particularly suitable for delivery of flow therapies, in which the RPT device generates the flow of air at controlled flow rates rather than controlled pressures. The “vent” at the unsealed patient interface 3800, through which excess airflow escapes to ambient, is the passage between the end of the prongs 3810a and 3810b of the cannula 3800 via the patient’s nares to atmosphere.

### 5.4 Rpt Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient’s airways, such as to treat one or more of the respiratory conditions described elsewhere in the present document.

In one form, the RPT device 4000 is constructed and arranged to be capable of delivering a flow of air in a range of -20 L/min to +150 L/min while maintaining a positive pressure of at least 6 cmH₂O, or at least 10 cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274. One or more of the air path items may be located within a removable unitary structure which will be referred to as a pneumatic block 4020. The pneumatic block 4020 may be located within the external housing 4010. In one form a pneumatic block 4020 is supported by, or formed as part of, the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110. In one form, an inlet air filter 4112 is located at the beginning of the pneumatic path upstream of a pressure generator 4140. In one form, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120. In one form of the present technology, an inlet muffler 4122 is located in the pneumatic path upstream of a pressure generator 4140. In one form of the present technology, an outlet muffler 4124 is located in the pneumatic path between the pressure generator 4140 and a patient interface 3000 or 3800.

### 5.4.1.3 Pressure or Flow Generator

In certain forms of the technology, the RPT device 4000 comprises a pressure generator or flow generator 4140. In one form of the present technology, a pressure or flow generator 4140 for producing a flow, or a supply, of air at positive pressure is a controllable blower 4142. For example the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O when delivering respiratory pressure therapy. The blower may be as described in any one of the following patents or patent applications the contents of which are incorporated herein by reference in their entirety: U.S. Pat. No. 7,866,944; U.S. Pat. No. 8,638,014; U.S. Pat. No. 8,636,479; and PCT Patent Application Publication No. WO 2013/020167.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of noncontact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800. In examples, the one or more transducers 4270 may comprise a flow rate sensor 4274 (e.g. based on a differential pressure transducer, for example, an SDP600 Series differential pressure transducer from SENSIRION), a pressure sensor 4272 located in fluid communication with the pneumatic path (for example, a transducer from the HONEYWELL ASDX series, or a transducer from the NPA Series from GENERAL ELECTRIC), and/or a motor speed transducer 4276 used to determine a rotational velocity of the motor 4144 and/or the blower 4142 (for example, a speed sensor, such as a Hall effect sensor). In other examples the one or more transducers 4270 may comprise an acoustic sensor (e.g. a microphone) and/or an optical sensor (e.g. a camera or barcode reader).

In one form, a signal from a transducer 4270 may be filtered, such as by low-pass, high-pass or band-pass filtering.

### 5.4.2 RPT Device Electrical Components

### 5.4.2.1 Power Supply

A power supply 4210 may be located internal or external of the external housing 4010 of the RPT device 4000. In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person (for example a patient, clinician or caregiver) to interact with the device. The buttons, switches or dials may be physical devices, or software devices accessible via a touch screen. The buttons, switches or dials may, in one form, be physically connected to the external housing 4010. In one form of the technology an input device 4220 may take the form of a keypad or keyboard with buttons enabling a user to enter a string of characters, for example a series of alphanumeric characters. The keypad may be formed of physical buttons or regions of a touch screen device visually displayed as buttons, or a combination of such buttons.

In other forms, an input device 4220 may take the form of a remote external device 4286 and/or a local external device 4288 separate, or separable, from the RPT device 4000 and in wireless communication with a data communication interface 4280 of the RPT device 4000 that is in electrical connection to the central controller 4230. Exemplary types of wireless communication between the remote external device 4286 and/or a local external device 4288 and the data communication interface 4280 are stated further below.

In one form of the technology, the input device 4220 is a mobile computing device, for example a mobile phone. The mobile computing device may be operable to communicate directly or indirectly with the central controller 4230, for example via an intermediate communication device and/or via data communication interface 4280. The mobile computing device may be configured to run one or more software applications, or apps, that cause one or more graphical user interfaces (GUIs) to be displayed to a user on a screen of the mobile computing device.

In one form, the input device 4220 may be constructed and arranged to allow a person to select a value and/or a menu option.

In certain forms of the technology, one or more transducers 4270 may operate as input devices 4220 enabling information to be sent to central controller 4230. For example, information may be received acoustically (e.g. via multifrequency signalling) and this information may be input to the RPT device 4000 by detection of the acoustic signal by an acoustic sensor. In another example, information may be received optically (e.g. via barcode, QR code or coded flashing light) and this information may be input to the RPT device 4000 by detection of the optical signal by an optical sensor. It will be appreciated that the data communication interface 4280 may also comprise one or more transducers 4270 (e.g. antennae) and may act as another input device 4220 by which information can be sent to the central controller 4230.

The input devices 4220 are configured to generate signals representative of information or data input by a user and to send the signals to the central controller 4230. For example, the signals may be electrical signals sent along wired connections to the central controller 4230. Additionally, or alternatively, the signals may be wireless communication signals. In one form of the technology, a keypad generates data representative of a character string entered by a user into the keypad and sends data representative of the character string to the central controller 4230.

### 5.4.2.3 Central Controller

In one form of the present technology, the central controller 4230 is one or a plurality of processors suitable to control an RPT device 4000. Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit. In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000. The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Therapy Device Controller

In one form of the present technology, therapy device controller 4240 is a virtual controller in the form of therapy control module 4330 that forms part of the algorithms 4300 executed by the central controller 4230. In one form of the present technology, therapy device controller 4240 is a dedicated motor control integrated circuit. For example, in one form a MC33035 brushless DC motor controller, manufactured by ONSEMI is used.

### 5.4.2.5 Memory

In accordance with one form of the present technology the RPT device 4000 includes memory 4260, e.g., non-volatile memory. In some forms, memory 4260 may include battery powered static RAM. In some forms, memory 4260 may include volatile RAM. Memory 4260 may be located on the PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.6 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet. In one form, local external communication network 4284 utilises one or more communication standards, such as Bluetooth, Near-Field Communication (NFC) or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician. The local external device 4288 may be a personal computer, mobile computing device (for example a mobile phone or tablet) or remote control.

### 5.4.2.7 Output Devices Including Optional Display, Alarms

In forms of the technology, the RPT device 4000 includes one or more output devices 4290.

An output device 4290 in accordance with the present technology may take the form of one or more of a visual, audio and haptic unit. A visual display may be a Liquid Crystal Display (LCD) or Light Emitting Diode (LED) display (which may include, for example an Organic Light Emitting Diode (OLED) display and subtypes thereof, such as Passive-Matrix Organic Light Emitting Diode (PMOLED) and Active-Matrix Organic Light Emitting Diode (AMOLED) display).

In forms of the technology the output device 4290 may be comprised as part of a remote external device 4286 and/or a local external device 4288. For example, the output device 4290 may be a display on a mobile computing device (for example a mobile phone or tablet) in wireless communication with the central controller 4230. The mobile computing device may be configured to run one or more software applications, or apps, that cause information to be output on a screen of the mobile computing device.

Data communication interface 4280 may operate as another form of output device 4290 since it may enable information to be output from the RPT device 4000.

### 5.4.3 RPT Device Operating Parameters and Settings

The central controller 4230 may control operation of the RPT device 4000 in accordance with one or more operating parameters. Operating parameters are factors that influence the operation of the RPT device 4000. Each operating parameter can vary. Operating parameters may be quantitative and/or qualitative.

Operating parameters may relate to the respiratory therapy provided to the patient 1000 by RPT device 4000, which operating parameters may be referred to as therapy parameters. Operating parameters may also relate to other aspects of the operation of the RPT device 4000, for example humidification parameters relate to the humidification of air being supplied to a patient by the RPT device 4000, and input device parameters (e.g. graphical user interface (GUI) parameters) relate to input devices of the RPT device 4000.

For each operating parameter there may be one or more settings that the operating parameter may be able to take. Put another way, an operating parameter is a variable and the settings are possible ‘values’ for the respective operating parameter. The settings may be quantitative values, e.g. a numerical value of pressure or temperature, or qualitative ‘values’, which, as they are non-numerical, may be referred to as qualitative measures, e.g. a level or qualitative descriptor. Settings may be discrete or continuous.

The table below indicates, for RPT device 4000, a non-limiting list of exemplary aspects to which operating parameters may relate, exemplary operating parameters and exemplary possible settings for each operating parameter according to certain forms of the technology.

| Aspect | Operating Parameter | Setting |
|---|---|---|
| Therapy | Program | Program 1 |
| | | Program 2 |
| | | Program 3 |
| | | Program 4 |
| | Therapy Mode | CPAP |
| | | AutoSet |
| | | AutoSet for Her |
| | | Spont |
| | | Timed |
| | | ST |
| | | iVAPS |
| | | ASV |
| | | VAuto |
| | | PAC |
| | Set Pressure | Value of pressure (e.g. 2-50 cmH₂O) |
| | Start Pressure | Value of pressure (e.g. 2-50 cmH₂O) |
| | Inspiratory Pressure | Value of pressure (e.g. 2-50 cmH₂O) |
| | Expiratory Pressure | Value of pressure (e.g. 2-50 cmH₂O) |
| | Pressure Support | Value of pressure (e.g. 0-50 cmH₂O) |
| | Patient Height | Value of height (e.g. 110-250 cm) |
| | Easy Breathe | On |
| | | Off |
| | Respiratory Rate | Value of rate (e.g. 2-90 breaths per minute) |
| | Rise Time | Value of time (e.g. 100-900 ms) |
| | Fall Time | Value of time (e.g. 100-700 ms) |
| | Inspiratory Time | Value of time (e.g. 0.1-4 seconds) |
| | Trigger Type | None |
| | | Absolute |
| | | Relative |
| | Trigger Sensitivity | Off |
| | | Very Low |
| | | Low |
| | | Medium |
| | | High |
| | | Very High |
| | Cycle Type | Discrete |
| | | Continuous |
| | | Auto |
| | Cycle Sensitivity | Very Low |
| | | Low |
| | | Medium |
| | | High |
| | | Very High |
| Feature | Comfort | On |
| | | Off |
| | Expository Pressure | Off |
| | Relief (EPR) | 1 |
| | | 2 |
| | | 3 |
| | Auto Ramp | On |
| | | Off |
| | Smart Start/Stop | On |
| | | Off |
| | Safety Volume | Value of volume (e.g. 0.05 - 2.5 L) |
| Circuit / Peripheral device | Patient Interface Type | Full Face |
| | | Nasal |
| | | Pillows |
| | | Invasive |
| | | Non-vented |
| | Patient Interface Model | ResMed AirFit P10 |
| | | ResMed AirFit N10 |
| | | ResMed Mirage Swift™ LT |
| | | ResMed Mirage Activa™ |
| | Tube Type | 15 mm non-heated |
| | | 19 mm non-heated |
| | | 3 m non-heated |
| | | 15 mm heated |
| | | 19 mm heated |
| | Antibacterial Filter | Yes |
| | | No |
| Climate | Climate Control | Auto |
| | | Manual |
| | Humidifier Level | Level value (e.g. 1-8) |
| | Heated Tube Temperature | Value of temperature (e.g. 16-35° C.) |
| | External Humidifier | Yes |
| | | No |
| Patient | Patient Type | Adult |
| | | Paediatric |
| | Patient Pathology | Obstructive |
| | | Restrictive |
| | | OHS |
| | | Normal |
| Respiratory device | Airplane Mode | On |
| | | Off |
| | Language | English |
| | | French |
| | | German |
| | | Italian |
| | | Spanish |
| | | Portuguese |
| | | Dutch |
| | | Japanese |
| | | Etc... |
| | Screen Brightness | Value of brightness (e.g. 0-100%) |
| | Font size | Small |
| | | Medium |
| | | Large |
| | Interface colour theme | Blue |
| | | Red |
| | | Green |
| | Show advanced settings | On |
| | | Off |
| Alarm Conditions | Inspiratory pressure | Threshold value of pressure |
| | Airway pressure | Threshold value of pressure |
| | Respiratory rate | Threshold value of respiratory rate |
| | Pulse rate | Threshold value of pulse rate |
| | Leak | Threshold value of leak |

Some of the exemplary settings in the above table are indicated as values. In certain forms of the technology, operating parameters whose setting is a value may have a discrete number of possible settings. For example, the treatment pressure may be able to be set to discrete values of cmH₂O in a predetermined range (e.g. values of treatment pressure in increments of 0.2 cmH₂O in the range 3 to 20 cmH₂O). In such forms, there is a finite plurality of values for the setting.

In other forms, these operating parameters may be set to a continuous variable. Even as a continuous variable, forms of the technology may set limits on the setting. For example, the setting may have maximum and minimum possible values limiting the setting to a range of permissible values between the maximum and minimum, and there may be a limit to the number of significant figures for the variable able to be set by the central controller 4230. Therefore, in such forms, again, there is a finite plurality of values for the setting.

Selection of settings for any one or more of the operating parameters in accordance with which the central controller 4230 controls operation of the RPT device 4000 may be effected in order to, at least in part, configure the RPT device 4000. In certain forms of the technology, configuration of the RPT device 4000 by selecting settings for operating parameters may be effected manually by a person (e.g. patient, clinician). For example, settings may be input to the central controller 4230 through manual input into input devices 4220. Additionally, or alternatively, configuration of the RPT device 4000 by selecting settings for operating parameters may be effected automatically by the central controller 4230. For example, settings may be input to the central controller 4230 from transducers 4270 and/or input devices 4220, and/or settings may be determined by one or more algorithms 4300 implemented by the central controller 4230.

The RPT device 4000 may be configured to, for one or more of the operating parameters, adopt one of the settings by default in the absence of input or determination of a setting for that/those operating parameter(s). The default setting for each operating parameter may be able to be changed based on manual input to the input devices 4220. Alternatively a default setting for one or more operating parameters may be able to be determined automatically by the central controller 4230 using an algorithm.

### 5.4.4 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and / or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.4.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.4.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea / hypopnea determination 4325, snore determination 4326, airway patency determination 4327, target ventilation determination 4328, and therapy parameter determination 4329.

In forms of the technology, the therapy engine module 4320, and any one or more of the algorithms thereof, may use any one or more of the settings of the operating parameters as inputs.

In forms of the technology, the therapy engine module 4320, and any one or more of the algorithms thereof, may provide any one or more of the settings of the operating parameters as outputs.

### 5.4.4.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.4.4 Engine and Control Module for Other Operating Parameters

It has been explained that the central controller 4230 may be configured to implement one or more algorithms 4300 for controlling delivery of respiratory therapy, the algorithms being grouped into a pre-processing module 4310, a therapy engine module 4320 and a therapy control module 4330. The central controller 4230 may additionally, or alternatively, be configured to implement one or more algorithms 4300 for controlling other aspects of the operation of the RPT device 4000. The one or more algorithms 4300 for controlling other aspects of the operation of the RPT device 4000 may be grouped into a pre-processing module, an operation engine module and an operation control module.

In forms of the technology, the operation engine module 4320, and any one or more algorithms thereof, may use any one or more of the settings of the operating parameters as inputs, and/or may provide any one or more of the settings of the operating parameters as outputs.

The other aspects of operation of the RPT device 4000 that may be controlled by algorithms 4300 include humidification and input devices (e.g. a graphical user interface (GUI)).

### 5.4.4.5 Detection of Fault Conditions

In one form of the present technology, the central controller 4230 executes one or more methods 4340 for the detection of fault conditions, for example, power failure (no power, or insufficient power), transducer fault detection, failure to detect the presence of a component, operating parameters outside recommended ranges (e.g. pressure, flow rate, temperature, PaO₂), and failure of a test alarm to generate a detectable alarm signal.

Upon detection of the fault condition, the corresponding algorithm 4340 signals the presence of the fault by one or more of the following: initiation of an audible, visual &/or kinetic (e.g. vibrating) alarm, sending a message to an external device, and logging of the incident.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800. In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used. In some forms, the air circuit 4170 may comprise one or more heating elements configured to heat air in the air circuit, for example to maintain or raise the temperature of the air. The heating element may be in communication with a controller such as a central controller 4230.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 Humidifier

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient’s airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. In one form, the humidifier 5000 may comprise a humidifier reservoir dock 5130 (as shown in FIG. 5B) configured to receive the humidifier reservoir 5110.

The water reservoir 5110 may be configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building’s water supply system.

The humidifier 5000 may comprise one or more humidifier transducers (sensors) 5210 instead of, or in addition to, transducers 4270 described above. Humidifier transducers 5210 may include one or more of an air pressure sensor 5212, an air flow rate transducer 5214, a temperature sensor 5216, or a humidity sensor 5218 as shown in FIG. 5C. A humidifier transducer 5210 may produce one or more output signals which may be communicated to a controller such as the central controller 4230 and/or the humidifier controller 5250. In some forms, a humidifier transducer may be located externally to the humidifier 5000 (such as in the air circuit 4170) while communicating the output signal to the controller.

A heating element 5240 may be provided to the humidifier 5000 in some cases to provide a heat input to one or more of the volume of water in the humidifier reservoir 5110 and/or to the flow of air.

According to one arrangement of the present technology, a humidifier 5000 may comprise a humidifier controller 5250 as shown in FIG. 5C. In one form, the humidifier controller 5250 may be a part of the central controller 4230. In another form, the humidifier controller 5250 may be a separate controller, which may be in communication with the central controller 4230.

In one form, the humidifier controller 5250 may receive as inputs measures of properties (such as temperature, humidity, pressure and/or flow rate), for example of the flow of air, the water in the reservoir 5110 and/or the humidifier 5000. The humidifier controller 5250 may also be configured to execute or implement humidifier algorithms and/or deliver one or more output signals.

As shown in FIG. 5C, the humidifier controller 5250 may comprise one or more controllers, such as a central humidifier controller 5251, a heated air circuit controller 5254 configured to control the temperature of a heated air circuit 4171 and/or a heating element controller 5252 configured to control the temperature of a heating element 5240.

### 5.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak -0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Screening, Diagnosis, Monitoring Systems

### 5.8.1 Polysomnography

FIG. 7A shows a patient 1000 undergoing polysomnography (PSG). A PSG system comprises a headbox 2000 which receives and records signals from the following sensors: an EOG electrode 2015; an EEG electrode 2020; an ECG electrode 2025; a submental EMG electrode 2030; a snore sensor 2035; a respiratory inductance plethysmogram (respiratory effort sensor) 2040 on a chest band; a respiratory inductance plethysmogram (respiratory effort sensor) 2045 on an abdominal band; an oro-nasal cannula 2050 with oral thermistor; a photoplethysmograph (pulse oximeter) 2055; and a body position sensor 2060. The electrical signals are referred to a ground electrode (ISOG) 2010 positioned in the centre of the forehead.

### 5.8.2 Non-Obtrusive Monitoring System

One example of a monitoring apparatus 7100 for monitoring the respiration of a sleeping patient 1000 is illustrated in FIG. 7B. The monitoring apparatus 7100 contains a contactless motion sensor generally directed toward the patient 1000. The motion sensor is configured to generate one or more signals representing bodily movement of the patient 1000, from which may be obtained a signal representing respiratory movement of the patient.

### 5.8.3 Respiratory Polygraphy

Respiratory polygraphy (RPG) is a term for a simplified form of PSG without the electrical signals (EOG, EEG, EMG), snore, or body position sensors. RPG comprises at least a thoracic movement signal from a respiratory inductance plethysmogram (movement sensor) on a chest band, e.g. the movement sensor 2040, a nasal pressure signal sensed via a nasal cannula, and an oxygen saturation signal from a pulse oximeter, e.g. the pulse oximeter 2055. The three RPG signals, or channels, are received by an RPG headbox, similar to the PSG headbox 2000.

In certain configurations, a nasal pressure signal is a satisfactory proxy for a nasal flow rate signal generated by a flow rate transducer in-line with a sealed nasal mask, in that the nasal pressure signal is comparable in shape to the nasal flow rate signal. The nasal flow rate in turn is equal to the respiratory flow rate if the patient’s mouth is kept closed, i.e. in the absence of mouth leaks.

FIG. 7C is a block diagram illustrating a screening / diagnosis / monitoring device 7200 that may be used to implement an RPG headbox in an RPG screening / diagnosis / monitoring system. The screening / diagnosis / monitoring device 7200 receives the three RPG channels mentioned above (a signal indicative of thoracic movement, a signal indicative of nasal flow rate, and a signal indicative of oxygen saturation) at a data input interface 7260. The screening / diagnosis / monitoring device 7200 also contains a processor 7210 configured to carry out encoded instructions. The screening / diagnosis / monitoring device 7200 also contains a non-transitory computer readable memory / storage medium 7230.

Memory 7230 may be the screening / diagnosis / monitoring device 7200′s internal memory, such as RAM, flash memory or ROM. In some implementations, memory 7230 may also be a removable or external memory linked to screening / diagnosis / monitoring device 7200, such as an SD card, server, USB flash drive or optical disc, for example. In other implementations, memory 7230 can be a combination of external and internal memory. Memory 7230 includes stored data 7240 and processor control instructions (code) 7250 adapted to configure the processor 7210 to perform certain tasks. Stored data 7240 can include RPG channel data received by data input interface 7260, and other data that is provided as a component part of an application. Processor control instructions 7250 can also be provided as a component part of an application program. The processor 7210 is configured to read the code 7250 from the memory 7230 and execute the encoded instructions. In particular, the code 7250 may contain instructions adapted to configure the processor 7210 to carry out methods of processing the RPG channel data provided by the interface 7260. One such method may be to store the RPG channel data as data 7240 in the memory 7230. Another such method may be to analyse the stored RPG data to extract features. The processor 7210 may store the results of such analysis as data 7240 in the memory 7230.

The screening / diagnosis / monitoring device 7200 may also contain a communication interface 7220. The code 7250 may contain instructions configured to allow the processor 7210 to communicate with an external computing device via the communication interface 7220. The mode of communication may be wired or wireless. In one such implementation, the processor 7210 may transmit the stored RPG channel data from the data 7240 to the remote computing device. In such an implementation, the remote computing device may be configured to analyse the received RPG data to extract features. In another such implementation, the processor 7210 may transmit the analysis results from the data 7240 to the remote computing device.

Alternatively, if the memory 7230 is removable from the screening / diagnosis / monitoring device 7200, the remote computing device may be configured to be connected to the removable memory 7230. In such an implementation, the remote computing device may be configured to analyse the RPG data retrieved from the removable memory 7230 to extract the features.

### 5.9 Portable Oxygen Concentrator

Oxygen concentrators may take advantage of pressure swing adsorption (PSA). Pressure swing adsorption may involve using a compressor to increase gas pressure inside a canister that contains particles of a gas separation adsorbent. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. Published Pat. Application No. 2009-0065007, published Mar. 12, 2009, and entitled “Oxygen Concentrator Apparatus and Method”, which is incorporated herein by reference.

FIG. 8A illustrates a schematic diagram of an oxygen concentrator 8000, according to an implementation. Oxygen concentrator 8000 may concentrate oxygen out of an air stream to provide oxygen enriched gas to a user. Oxygen concentrator 8000 may be a portable oxygen concentrator. For example, oxygen concentrator 8000 may have a weight and size that allows the oxygen concentrator to be carried by hand and/or in a carrying case.

Oxygen may be collected from ambient air by pressurising ambient air in canisters 8100, including first canister 8102 and 8104, which include a gas separation adsorbent. Gas separation adsorbents useful in an oxygen concentrator are capable of separating at least nitrogen from an air stream to produce oxygen enriched gas. As shown in FIG. 8B, air may be drawn into the oxygen concentrator 8000 through air inlet 8002 by compression system 8200. Compression system 8200 may draw in air from the surroundings of the oxygen concentrator and compress the air, forcing the compressed air into one or both of canisters 8102 and 8104. Compression system 8200 may include one or more compressors capable of compressing air. In an implementation, an inlet muffler 8004 may be coupled to air inlet 8002 to reduce sound produced by air being pulled into the oxygen concentrator by compression system 8200.

Coupled to each canister 8102/8104 are inlet valves 8020/8022 and outlet valves 8030/8032. As shown in FIG. 8B, inlet valves 8020/8022 are used to control the passage of air from compression system 8200 to the respective canisters. Outlet valves 8030/8032 are used to release gas from the respective canisters during a venting process. In an implementation, pressurized air is sent into one of canisters 8102 or 8104 while the other canister is being vented.

In an implementation, a controller 8300 is electrically coupled to valves 8020, 8022, 8030, and 8032. Controller 8300 includes one or more processors 8310 operable to execute program instructions stored in memory 8320. The program instructions are operable to perform various predefined methods that are used to operate the oxygen concentrator 8000, such as the methods described in more detail herein. Controller 8300 may include program instructions for operating inlet valves 8020 and 8022 out of phase with each other, i.e., when one of inlet valves 8020 or 8022 is opened, the other valve is closed. During pressurization of canister 8102, outlet valve 8030 is closed and outlet valve 8032 is opened. Similar to the inlet valves, outlet valves 8030 and 8032 are operated out of phase with each other. In some implementations, the voltages and the duration of the voltages used to open the input and output valves may be controlled by controller 8300.

Check valves 8040 and 8042 are coupled to canisters 8102 and 8104, respectively. Check valves 8040 and 8042 are one-way valves that are passively operated by the pressure differentials that occur as the canisters are pressurized and vented. Check valves 8040 and 8042 are coupled to canisters to allow oxygen produced during pressurization of the canister to flow out of the canister, and to inhibit back flow of oxygen or any other gases into the canister.

Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In an exemplary implementation, canister 8102 is pressurized by compressed air produced in compression system 8200 and passed into canister 8102, and canister 8104 is vented substantially simultaneously while canister 8102 is pressurized. Canister 8102 is pressurized until the pressure in canister is sufficient to open check valve 8040. Oxygen enriched gas produced in canister 8102 exits through check valve and, in one implementation, is collected in accumulator 8006.

After some time, the gas separation adsorbent will become saturated with nitrogen and will be unable to separate significant amounts of nitrogen from incoming air. This point is usually reached after a predetermined time of oxygen enriched gas production. In the implementation described above, when the gas separation adsorbent in canister 8102 reaches this saturation point, the inflow of compressed air is stopped and canister 8102 is vented to remove nitrogen. While canister 8102 is being vented, canister 8104 is pressurized to produce oxygen enriched gas in the same manner described above. Pressurization of canister 8104 is achieved by closing outlet valve 8032 and opening inlet valve 8022. The oxygen enriched gas exits canister 8104 through check valve 8042.

During venting of canister 8102, outlet valve 8030 is opened allowing pressurized gas (mainly nitrogen) to exit the canister through concentrator outlet 8008. In an implementation, the vented gases may be directed through muffler 8010 to reduce the noise produced by releasing the pressurized gas from the canister. As gas is released from canister 8102, the pressure in the canister drops, allowing the nitrogen to become desorbed from the gas separation adsorbent. The released nitrogen exits the canister through outlet 8008, resetting the canister to a state that allows renewed separation of oxygen from an air stream.

During venting of the canisters, it is advantageous that at least a majority of the nitrogen is removed. In some implementations, a canister may be further purged of nitrogen using an oxygen enriched stream that is introduced into the canister from the other canister. In an exemplary implementation, a portion of the oxygen enriched gas may be transferred from canister 8102 to canister 8104 when canister 8104 is being vented of nitrogen. In an implementation, oxygen enriched gas may travel through flow restrictors 8050, 8052, and 8054 between the two canisters.

Flow of oxygen enriched gas is also controlled by use of valve 8056 and valve 8058. Valves 8056 and 8058 may be opened for a duration during the venting process (and may be closed otherwise) to prevent excessive oxygen loss out of the purging canister. In an exemplary implementation, when canister 8102 is being vented it is desirable to purge canister 8102 by passing a portion of the oxygen enriched gas being produced in canister 8104 into canister 8102. A portion of oxygen enriched gas, upon pressurization of canister 8104, will pass through flow restrictor 8050 into canister 8102 during venting of canister 8102. Additional oxygen enriched air is passed into canister 8102, from canister 8104, through valve 8058 and flow restrictor 8054. Valve 8056 may remain closed during the transfer process, or may be opened if additional oxygen enriched gas is needed. The selection of appropriate flow restrictors 8050 and 8054, coupled with controlled opening of valve 8058 allows a controlled amount of oxygen enriched gas to be sent from canister 8104 to 8102. In an implementation, the controlled amount of oxygen enriched gas is an amount sufficient to purge canister 8102 and minimize the loss of oxygen enriched gas through venting valve 8030 of canister 8102. While this implementation describes venting of canister 8102, it should be understood that the same process can be used to vent canister 8104 using flow restrictor 8050, valve 8056 and flow restrictor 8052.

The pair of equalization/vent valves 8056/8058 work with flow restrictors 8052 and 8054 to optimize the air flow balance between the two canisters. In some implementations, the air pathway may not have restrictors but may instead have a valve with a built-in resistance or the air pathway itself may have a narrow radius to provide resistance.

At times, oxygen concentrator may be shut down for a period of time. In an implementation, outside air may be inhibited from entering canisters after the oxygen concentrator is shutdown by pressurising both canisters prior to shutdown. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa).

FIG. 8B depicts an example of the portable oxygen concentrator 8000. In this implementation the oxygen concentrator 8000 includes an outer housing 8500. Outer housing 8500 includes compression system inlets 8502, cooling system passive inlet 8504 (not seen in FIG. 8B, but indicated by reference numeral 8502) and cooling system passive outlet 8504 at each end of outer housing 8500. The outer housing 8500 also includes outlet port 8506, and a control panel 8600. Inlet 8502 and outlet 8504 allow cooling air to enter the housing 8500, flow through the housing, and exit the interior of housing to aid in cooling of the oxygen concentrator 8000. Compression system inlets 8502 allow air to enter the compression system 8200 shown in FIG. 8A). Outlet port 8506 is used to attach a conduit to provide oxygen enriched gas produced by the oxygen concentrator 8000 to a user. Control panel 8600 serves as an interface between a user and controller 8300 (shown in FIG. 8A) to allow the user to initiate predetermined operation modes of the oxygen concentrator 8000 and to monitor the status of the system. Charging input port 8602 may be disposed in control panel 8600.

### 5.10 Respiratory Therapy Modes

Various respiratory therapy modes may be implemented by the disclosed respiratory therapy system. Examples of the respiratory therapy modes may include CPAP therapy, bi-level therapy, and high flow therapy.

### 5.11 Computing System and Processes

In forms of the technology, the RPT device 4000 may be part of, or may operate in conjunction with, a system 9000. System 9000 may comprise one or more servers 9010 and one or more computing devices 9040, and may generally be referred to as a computing system 9000. Components of system 9000 may interact with RPT device 4000, for example to control and/or monitor operation of the RPT device 4000. In some examples, system 9000 may enable a person (e.g. a patient, a clinician) to control and/or monitor operation of the RPT device 4000. Controlling and/or monitoring operation of the RPT device 4000 may enable the respiratory therapy provided to the patient 1000 to be controlled and/or monitored.

### 5.11.1 Computing System

FIG. 9 depicts an example system 9000 in accordance with certain forms of the technology. The system 9000 may generally include one or more of servers 9010, one or more communication networks 9030, and one or more computing devices 9040. The server 9010 and computing device 9040 may also be in communication with one or more respiratory therapy devices (for example, but not limited to, the RPT device 4000 described in relation to FIG. 4A to FIG. 4F above) via the one or more communication networks 9030.

The one or more communication networks 9030 may comprise, for example, the Internet, a local area network, a wide area network and/or a personal area network implemented over wired communication network(s) 9032, wireless communication network(s) 9034, or a combination thereof (for example, a wired network with a wireless link). In one form, local communication networks may utilize one or more communication standards, such as Bluetooth, Near-Field Communication (NFC) or a consumer infrared protocol.

The server 9010 may comprise processing facilities represented by one or more processors 9012, memory 9014, and other components typically present in such computing environments. The processing capabilities of the processor 9012 may be provided, for example, by one or more general-purpose processors, one or more special-purpose processors, or cloud computing services providing access to a shared pool of computing resources configured in accordance with desired characteristics, service models, and deployment models. In the example illustrated the memory 9014 stores information accessible by processor 9012, the information including instructions 9016 that may be executed by the processor 9012 and data 9018 that may be retrieved, manipulated or stored by the processor 9012. The memory 9014 may be of any suitable means known in the art, capable of storing information in a manner accessible by the processor 9012, including a computer readable medium, or other medium that stores data that may be read with the aid of an electronic device. Although the processor 9012 and memory 9014 are illustrated as being within a single unit, it should be appreciated that this is not intended to be limiting, and that the functionality of each as herein described may be performed by multiple processors and memories, that may or may not be remote from each other and the remainder of system 9000.

The instructions 9016 may include any set of instructions suitable for execution by the processor 9012. For example, the instructions 9016 may be stored as computer code on the computer readable medium. The instructions may be stored in any suitable computer language or format. Data 9018 may be retrieved, stored or modified by processor 9012 in accordance with the instructions 9016. The data 9018 may also be formatted in any suitable computer readable format. Again, while the data is illustrated as being contained at a single location, it should be appreciated that this is not intended to be limiting - the data may be stored in multiple memories or locations. The data 9018 may include one or more databases 9020.

In some examples, the server 9010 may communicate one-way with computing device(s) 9040 by providing information to one or more of the computing devices 9040, or vice versa. In other embodiments, server 9010 and computing device(s) 9040 may communicate with each other two-way and may share information and/or processing tasks.

In some examples, the computing device(s) 9040 may include the remote external device 4286 and/or the local external device 4288 described with reference to FIG. 4C above.

### 5.11.2 Computing Devices

The computing device(s) 9040 can be any suitable processing device such as, without limitation, a personal computer such as a desktop or laptop computer 9042, or a mobile computing device such as a smartphone 9044 or tablet 9046. FIG. 10 depicts an exemplary general architecture 9100 of a computing device 9040. Computing device 9040 may include one or more processors 9110. Computing device 9040 may also include memory/data storage 9120, input/output (I/O) devices 9130, and communication interface 9150.

The one or more processors 9110 can include functional components used in the execution of instructions, such as functional components to fetch control instructions from locations such as memory/data storage 9120, decode program instructions, and execute program instructions, and write results of the executed instructions.

Memory/data storage 9120 may be the computing device’s internal memory, such as RAM, flash memory or ROM. In some examples, memory/data storage 9120 may also be external memory linked to computing device 130, such as an SD card, USB flash drive, optical disc, or a remotely located memory (e.g. accessed via a server such as server 9010), for example. In other examples, memory/data storage 9120 can be a combination of external and internal memory.

Memory/data storage 9120 includes processor control instructions 9122 and stored data 9124 that instruct processor 9110 to perform certain tasks, as described herein. As noted above, in examples instructions may be executed by, and data stored in and/or accessed from, resources associated with the server 9010 in communication with the computing device 9030.

In examples, the input/output (I/O) devices 9130 may include one or more displays 9132. In examples, the display 9132 may be a touch sensitive screen allowing for user input in addition to outputting visible information to a user of computing device 9030. In examples, I/O devices may include other output devices, including one or more speakers 9134, and haptic feedback devices 9136. In examples, the input/output (I/O) devices 9130 may include input devices such as physical input devices 9138 (for example, buttons or switches), optical sensors 9140 (for example, one or more imaging devices such as a camera), and inertial sensors 9142 (particularly in examples where the computing device 9030 is a mobile computing device). It will be appreciated that other I/O devices 9130 may be included, or otherwise accessed through an I/O interface 9150 (for example, interfacing with peripheral devices connected to the computing device 9130). A communication interface 9160 enables computing device 9030 to communicate via the one or more networks 9030 (shown in FIG. 9).

### 5.11.3 Computer-Implementable Methods

This specification includes flow diagrams indicating methods implementable, at least in part, by system 9000 in certain forms of the technology. The flow diagrams are representative of example computer readable instructions for implementing the exemplary methods. In examples, the computer readable instructions comprise one or more algorithms for execution by one or more of the processors, for example processors 9012 and/or central controller 4230, described herein. The instructions for performing these functions are, optionally, included in a non-transitory computer readable storage medium, for example memory 9014, or other computer program product configured for execution by one or more processors. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A computer readable storage medium, as used herein, is not to be construed as being transitory signals per se, such as radio waves or other freely propagating electromagnetic waves, electromagnetic waves propagating through a waveguide or other transmission media, or electrical signals transmitted through a wire.

However, persons of ordinary skill in the art will readily appreciate that the entire algorithm and/or parts thereof can alternatively be executed by a device other than a processor and/or embodied in firmware or dedicated hardware in a well-known manner, e.g., it may be implemented by an application specific integrated circuit (ASIC), a programmable logic device (PLD), a field programmable logic device (FPLD), a field programmable gate array (FPGA), discrete logic, etc. For example, any or all of the components can be implemented by software, hardware, and/or firmware. Also, some or all of the instructions represented by the flowcharts may be implemented manually. Further, although the example algorithms are described with reference to the illustrated flowcharts, persons of ordinary skill in the art will readily appreciate that many other methods of implementing the example processor readable instructions may alternatively be used. For example, the order of execution of the blocks may be changed, and/or some of the blocks described may be changed, eliminated, or combined.

As used herein the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor, a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a processor readable medium; or a combination thereof.

### 5.12 Configuration of Rpt Device Settings

Described earlier are problems that may be encountered, or may result, when a patient 1000, or other untrained user of the RPT device 4000, needs to configure the RPT device 4000 for use without supervision or consultation with a clinician, or other trained user of the RPT device 4000. In particular, one problem is the incorrect configuration of an RPT device 4000, which may result in patient 1000 receiving sub-optimal, ineffective or potentially harmful respiratory therapy.

To address these problems, certain forms of the technology provide methods and/or systems by which RPT device 4000 may be configured by selecting particular settings for any one or more of the operating parameters in accordance with which the central controller 4230 controls operation of the RPT device 4000. The particular settings selected may be selected for a specific patient 1000 in order to customise the RPT device 4000 to the particular therapy needs of the patient 1000.

### 5.12.1 Method of Configuring RPT Device

FIG. 11 is a flow diagram of a method 9500 of configuring RPT device 4000 according to certain forms of the technology. FIG. 11 illustrates parties that may be involved in the implementation of method 9500, which in the form shown includes clinician 1500, servers 9010, patient 1000 or local external device 4288, and central controller 4230 of RPT device 4000.

At step 9510 the clinician 1500 determines a plurality of settings that are suitable for provision of respiratory therapy for patient 1000. The settings determined by the clinician may be any settings of operating parameters of the RPT device 4000, as explained in section 8.4.3 above. Examples of settings and corresponding operating parameters are given in that section.

At step 9520 the clinician 1500 inputs the determined settings on a computing device 9040. The computing device 9040 may be configured to run a computer programme, or application, tailored to enable the clinician 1500 to enter the settings into the computing device 9040. For example, the application may present, on a display device of the computing device 9040, a screen presenting settings as a number of selectable values for each operating parameter. The clinician 1500 may select the appropriate setting value using an input device such as a mouse, keyboard and/or touchscreen. In some forms the settings may be presented as a drop-down list for selection by the clinician 1500. The application may present a mechanism by which the clinician 1500 may confirm the selection of settings, for example by selecting a “done” button.

At step 9530 the inputted settings are received by servers 9010. For example, data representative of the inputted settings may be sent from computing device 9040 to servers 9010, for example via network 9030.

At step 9540 servers 9010 determine an identifier from the settings. The identifier is information that may be used to identify the combination of settings selected by the clinician 1500. A further discussion of identifiers and how they may be used to identify the combination of settings is provided below.

At step 9550 the identifier determined at step 9540 is output by servers 9010. The identifier may be output in any one or more of a number of ways. For example, at step 9560 the identifier may be displayed to clinician 1500 on a display device of computing device 9040. Additionally, or alternatively, the identifier may be sent by the servers 9010 to another computing device 9040. In examples, the identifier is sent to a computing device of a patient, of another clinician or of a healthcare facility. In one form of the technology, the identifier is first sent to clinician 1500, for example by display on the computing device 9040 of the clinician 1500, and at step 9570 the clinician sends the identifier to another party. For example, the clinician 1500 may send the identifier to the patient 1000. In one form the clinician 1500 sends the identifier from the computing device 9040 of the clinician 1500 to the computing device 9040 of the patient 1000 over network 9030. The identifier may be sent by any suitable communications protocol including email, SMS, FTP, HTTP, HTTPS. In other forms, the clinician may send the identifier to the patient via another means of communication, for example, orally or by mail. In one form, the computing device of the patient 1000 is a local external device 4288 in communication with RPT device 4000.

At step 9580 patient 1000 and/or local external device 4288 and/or another computing device 9040 associated with the patient 1000, as the case may be, receives the identifier.

At step 9590 the identifier is input into RPT device 4000. The manner in which this step occurs depends on the nature of the identifier, and examples according to certain forms of the technology are described in more detail below. Generally, the identifier is input into RPT device 4000 via one or more of: data communication interface 4280; input devices 4220; and transducers 4270.

At step 9610 the central controller 4230 of RPT device 4000 receives the identifier. For example, data representative of the identifier may be sent from data communication interface 4280, input devices 4220 and/or transducers 4270 to central controller 4230.

At step 9620 the central controller 4230 determines, from the identifier, the settings for the RPT device 4000 that were determined by the clinician 1500 at step 9510 as being suitable for provision of respiratory therapy for patient 1000. A further discussion of how the identifier may be used to identify the combination of settings determined by the clinician 1500 is provided below.

At step 9630 the central controller 4230 configures the RPT device 4000 to operate in accordance with the combination of settings determined in step 9620. Configuration of the RPT device 4000 may be effected by implementation of therapy control module 4330 and/or another operation control module. Central controller 4230 may also store the combination of settings with which the RPT device 4000 is currently configured in memory 4260. Memory 4260 may also store combinations of settings used to configure the RPT device 4000 in the past.

The method illustrated in FIG. 11 allows RPT device 4000 to be configured for use with a specific patient 1000 in accordance with settings for operating parameters selected by a clinician 1500 or other healthcare professional without the clinician 1500 or healthcare professional being present, consulted with or configuring the RPT device 4000 themselves. What is required is that the identifier, which effectively encodes the operating settings, be communicated to the RPT device 4000 so that the central controller 4230 of the RPT device 4000 can configure the RPT device 4000 appropriately. In circumstances where a clinician 1500 or other healthcare professional is not available to help a patient 1000 set or up configure their RPT device 4000 by interacting with the RPT device directly, this method reduces the risk of the patient 1000 setting up the RPT device incorrectly and consequently receiving sub-optimal, ineffective or potentially harmful respiratory therapy.

### 5.12.2 Identifier and Combination of Settings Determination

In certain forms of the technology the central controller 4230 controls operation of the RPT device 4000 in accordance with a plurality of operating parameters O₁, O₂, O₃, ... Oₙ. Examples of operating parameters of the RPT device 4000 are given in the table in section 8.4.3 above.

Each of the operating parameters Oₓ may be set to any setting S^{Ox}ₓ out of a plurality of settings S^{Ox}₁, S^{Ox}₂, S^{Ox}₃, ... S^{Ox}ₓ. That is, the operating parameter O₁ may be set to any the settings S^{O1}₁, S^{O1}₂, S^{O1}₃, ... S^{O1}ᵢ and the operating parameter O₂ may be set to any the settings S^{O2}₁, S^{O2}₂, S^{O2}₃, ... S^{O2}ⱼ, etc. It is noted that the number of possible settings may not be the same for each operating parameter, hence i,j, etc may take different values. Examples of settings of the RPT device 4000 are given in the table in section 8.4.3 above.

Since there exists a finite number n of operating parameters Oₓ, each with a finite number mₓ = i,j, ... of possible settings S^{Ox}ₓ, there exists a finite number of combinations of settings {S^{O1}ₐ, S^{O2}_{b}, S^{O3}_{c}, ... S^{On}ₓ} for all the operating parameters Oₓ.

Therefore it is possible to define an identifier Iₓ for each possible combination of settings {S^{O1}ₐ, S^{O2}_{b}, S^{O3}_{c}, ... S^{On}ₓ}, and in doing so there is a finite plurality of identifiers Iₓ. In certain forms of the technology the combination of settings corresponding to each of the identifiers Iₓ is unique to each identifier. In other forms, the same combination of settings may correspond to more than one identifier.

In practice, it may not be necessary for all possible combinations of all the possible settings of each operating parameter to correspond to an identifier Iₓ. For example, some combinations of settings may not be practical or safe for operating RPT device 4000. In addition, one or more settings for a particular operating parameter may be incompatible with one or more settings of another operating parameter. The RPT device 4000 would not be configured with settings that are mutually incompatible, in which case no identifier is needed for combinations in which the incompatible settings are present. Therefore, the number of identifiers may be less than the number of combinations given by the product of the number of settings for each parameter, i.e. ij....

Forms of the technology provide an identifier determination algorithm for determining an identifier Iₓ from the corresponding combination of settings at step 9540. For example, servers 9010 are configured to receive a combination of settings as an input, perform the identifier determination algorithm, and output the identifier Iₓ corresponding to the inputted combination of settings.

Forms of the technology further provide a combination of settings determination algorithm for determining a combination of settings from the corresponding identifier Iₓ at step 9620. For example, central controller 4230 is configured to receive identifier Iₓ as an input, perform the combination of settings determination algorithm, and output the combination of settings corresponding to the identifier Iₓ. In forms of the technology where the same combination of settings may correspond to more than one identifier, either of the identifiers corresponding to that combination of settings may be output. The combination of settings determination algorithm is inverse to the identifier determination algorithm.

### 5.12.2.1 Data Array

In one form of the technology the identifier determination algorithm and/or the combination of settings determination algorithm are implemented using a data array 9700. FIG. 12 is a diagram of an exemplary data array 9700 for performing an identifier determination algorithm and/or a combination of settings determination algorithm according to forms of the technology. Data array 9700 may be alternatively referred to as a look-up table.

Data array 9700 is a memory store in which identifiers Iₓ are stored in correspondence to a setting S^{Ox}ₓ for each of a plurality of operating parameters Oₓ. In FIG. 12 this correspondence is shown as identifiers and combinations of settings occupying the same rows. Every combination of settings according to which the RPT device 4000 may need to be configured is represented in a row, and each row is allocated an identifier Iₓ. To perform the identifier determination algorithm, or the combination of settings determination algorithm, a look-up function is performed whereby the input combination of settings, or the input identifier, is identified in the data array 9700, and the corresponding identifier, or combination of settings, is identified from the relevant row.

### 5.12.2.2 Other Determination Algorithms

In certain forms of the technology the identifier may encode the combination of settings in some way. In such forms, the identifier determination algorithm and the combination of settings determination algorithm are algorithms that perform the encoding and decoding process respectively.

In one form, the identifier Iₓ may comprise a plurality of constituent identifier parts Iₓ₁, Iₓ₂, ... Iₓₙ, each constituent identifier part Iₓᵢ corresponding to one of the operating parameters Oₓ and having a value or representation indicative of the setting S^{Ox}ₓ for the respective operating parameter. For example, the identifier Iₓ may be a character string formed by concatenating the plurality of constituent identifier parts Iₓ₁, Iₓ₂, ... Iₓₙ. The concatenation may demarcate the constituent identifier parts, for example by using a separation indicator, e.g. “;”. Each constituent identifier part may represent the setting for the respective operating parameter by means of a data array or look-up table.

In other forms the identifier determination algorithm and the combination of settings determination algorithm may be any algorithm and its inverse enabling a combination of settings to be uniquely represented by an identifier. In some forms, a hash function and its inverse may be used, for example.

### 5.12.2.3 Local or Remote Determination Algorithms

To perform the identifier determination algorithm at step 9540, servers 9010 may need to have access to a memory on which the steps of, or data relevant to perform, the identifier determination algorithm are stored. Similarly, to perform the combination of settings determination algorithm at step 9620, central controller 4230 may need to have access to a memory on which the steps of, or data relevant to perform, the combination of settings determination algorithm are stored. The different options for where these memories are located provide certain advantages.

Referring to the case of the identifier determination algorithm and the combination of settings determination algorithm being implemented using data array 9700 by way of non-limiting example, in one form of the technology, data array 9700 is stored in both memory 4260 of RPT device 4000 and in a memory accessible to servers 9010, for example memory 9014 or a memory accessible by servers 9010 via network 9030. This situation has the advantage that RPT device 4000 does not need to have remote communication capability to be able to determine the combination of settings selected by clinician 1500 from the received identifier, and therefore be configured according to the clinician’s intentions. In some cases it may not be practical or possible for RPT device 4000 to communicate remotely, for example if the RPT device 4000 does not have remote communication capabilities or if sufficiently strong data communication signals are not available to connect the RPT device 4000 to a data communication network. Any changes to data array 9700 may be reflected by updating the data array 9700 stored in memory 4260 on RPT device 4000, for example by way of a software update. For RPT devices 4000 not capable of remote communication, a portable memory device, for example a memory card made in accordance with the Secure Digital (SD) standard, may be sent to patient 1000 by mail for insertion into RPT device 4000 in order to implement the software update to update data array 9700. Similarly, existing RPT devices 4000 may be able to be upgraded so that they may be configured remotely according to method 9500 by physically sending a portable memory device provision on which is stored data array 9700 to a patient 1000 for insertion into RPT device 4000.

In another form, data array 9700 is stored in a memory located remote from one or both central controller 4230 and servers 9010, but accessible by both central controller 4230 and servers 9010, for example over data communication interface 4280 and network 9030. Storing the data array 9700 in a single location that is accessible to both RPT device 4000 and system 9000 means any updates to data array 9700 can be made once in the single location, but requires RPT device 4000 and system 9000 to both have remote communication abilities. In the case of RPT device 4000 in particular, this may not always be practical or desirable.

### 5.12.3 Identifier

An identifier according to forms of the technology may be any data or element that has the capability of identifying a combination of settings, for example upon the application of an appropriate combination of settings determination algorithm on the identifier.

In one form of the technology an identifier is a character string. The character string may be a string of alphanumeric characters or a string of characters encoding other characters, e.g. according to the ASCII or Unicode systems. For example, one identifier may be the character string ABCDEF12345 while another identifier may be the character string GHIJKL67890. In one form of the technology an identifier has the capability of identifying the corresponding combination of settings, and vice versa, by virtue of data array 9700, as explained above. It will be apparent that the length of the character string used to represent the identifiers needs to be sufficiently long to allow at least one unique identifier to be allocated to each combination of settings. Clearly, the number of choices for each character in the character string is also a factor in determining the minimum necessary length of the character string.

To send an identifier from one party or device to another party or device, the identifier may be sent in the form of data representative of the identifier. Non-limiting examples of how an identifier may be represented are: bits of data representative of a character string; electromagnetic signals with characteristics (e.g. frequency) encoding a character string; optical data representative of an optically machine-readable code (e.g. QR code or barcode) which is representative of a character string; and acoustic data representative of a plurality acoustic tones which are representative of a character string. Known data communication techniques are used to send the data representative of the identifier in order to communicate the identifier from one party or device to another.

In certain forms of the technology, the data representative of the identifier may be encrypted. For example, the servers 9010 may perform a step of encrypting the identifier prior to outputting the identifier at step 9550. Similarly, the central controller 4230 may perform a decryption step after receiving the identifier at step 9610. Conventional encryption and decryption methods may be used. Encrypting the identifier may alleviate concerns regarding privacy of patient-specific information. For example, in some jurisdictions the selection of settings for operating parameters of a respiratory device may be considered to be patient confidential information, similar to a prescription, and consequently be subject to privacy laws. Measures such as the encryption of the identifiers described above may assist in meeting requirements for maintaining confidentiality of patient information.

### 5.12.4 Identifier Input

An identifier may be provided to RPT device 4000 through any one or more input devices 4220. Examples of input devices that enable data in different forms to be input into RPT device 4000 have been described earlier. In forms of the technology, these input devices 4220, and the methods described, may be used to input an identifier into RPT device 4000 at step 9590. In some forms, the input devices 4220 may be user input devices, i.e. devices by which a user (such as patient 1000) performs a manual process as part of a data input process, for example by pushing buttons on a keypad or holding a display device in a particular location for detection by a sensor.

In one example, a patient 1000 receives an identifier in the form of a character string, e.g. ABCDEF12345, via email or text message from their clinician 1500. The patient 1000 enters ‘ABCDEF12345’ on a keypad or keyboard, for example a keypad displayed on a touch screen device, of the RPT device 4000. As a consequence, data representative of the identifier ABCDEF12345 is sent to central controller 4230.

In another example, a patient 1000 receives an identifier in the form of a character string, e.g. ABCDEF 12345, via email or text message from their clinician 1500. The patient 1000 enters ‘ABCDEF 12345’ in a field displayed on a screen of a mobile computing device, for example the patient’s mobile phone, by an app running on the mobile computing device using a keypad of the mobile computing device. The mobile computing device may be a local external device 4288 in communication with central controller 4230 over a local external communication network 4284, and the mobile computing device sends data representative of the identifier ABCDEF12345 to the central controller 4230 over the local external communication network 4284, for example via Bluetooth, Near-Field Communication (NFC) or a consumer infrared protocol. The mobile computing device may send data directly to the central controller 4230 or indirectly, e.g. via a receiver or other intermediate component(s).

In another example, a patient 1000 receives an identifier represented in the form of a QR code from their clinician 1500. The QR code may be sent to the patient via email, text message or through an app running on a mobile computing device of the patient 1000. The QR code may be representative of an identifier, for example in the form of a character string, e.g. ABCDEF12345. The patient 1000 prompts their mobile computing device to display the QR code on a screen of the device and presents the screen displaying the QR code to a camera or other optical sensor on the RPT device 4000, which senses the QR code. Optical data representative of the QR code is thereby sent to central controller 4230, which is configured to determine the identifier from the optical data. In a similar example, the patient 1000 may use their mobile computing device to read the QR code, and thereby generate optical data representative of the QR code and/or the identifier represented by the QR code, and the mobile computing device sends the optical data and/or the identifier to the RPT device 4000, for example over the local external communication network 4284 as described above.

In another example a patient 1000 receives an identifier represented by audio data in an audio data file. The patient 1000 plays the audio data file on a mobile computing device in audible proximity to the RPT device 4000. An acoustic sensor (e.g. a microphone) on the RPT device 4000 detects the acoustic signal from the mobile computing device and central controller 4230 determines the identifier from data representative of the acoustic signal.

In a further example an identifier is encoded in a sequence of flashing lights that may be generated by a mobile computing device of a patient 1000. The identifier may be provided to central controller 4230 through detection of the sequence of flashing lights by an optical sensor of the RPT device 4000.

### 5.12.5 Combination of Settings Input

It has already been described how a clinician 1500 may enter a combination of settings into a computing device 9040 in order to provide a combination of settings to servers 9010 of system 9000 at step 9520. In other examples, a combination of settings may be provided to servers 9010 of system 9000 at step 9520 using methods equivalent to those described above for providing an identifier to central controller 4230 of an RPT device 4000, where suitable, at step 9590.

### 5.12.6 Validation of Identifier

In some forms of the technology, an identifier received by a patient 1000 may not give any indication to the patient as to the combination of settings that the identifier corresponds to (i.e. the identifier may not be in a form that is understandable to a human). Therefore, the patient 1000 may not be aware of whether they have received the appropriate identifier. Furthermore, where the patient 1000, or other person, needs to take some part in the process of providing the identifier to the central controller 4230 of the RPT device 4000, for example by entering a character string into a keypad, there is a risk of user error in entering an incorrect identifier. If the incorrect identifier entered is also an identifier that corresponds to a combination of settings, this creates a risk that the RPT device 4000 may be configured with a combination of settings other than intended by the clinician 1500. This could lead to the patient 1000 receiving ineffective, or potentially harmful, respiratory therapy.

In certain forms of the technology there is provided a method and/or system for validating an identifier received by the central controller 4230 of RPT device 4000.

### 5.12.6.1 Validation Data - First Embodiment

A method 9800 for validating an identifier received by the central controller 4230 of RPT device 4000 according to one form of the technology is illustrated in FIG. 13. Systems according to forms of the technology previously described are used to implement the method 9800 of FIG. 13.

In some forms of the technology the validation method 9800 shown in FIG. 13 forms part of the configuration method 9500 of FIG. 11. Therefore, FIG. 13 includes some of the same steps as are shown in FIG. 11. Where steps from FIG. 11 are omitted from FIG. 13 this is for ease of presentation in the figure and not because the steps do not occur in the validation method 9800.

At step 9540 the servers 9010 of system 9000 determine an identifier from the combination of settings received from the clinician 1500 at step 9530. The identifier is output at step 9550 and sent directly or indirectly to central controller 4230 of RPT device 4000 where it is received at step 9610, as described in relation to FIG. 11.

In addition, at step 9810, servers 9010 generate validation data from the identifier determined at step 9540. The validation data is generated by applying a validation data generation algorithm to the identifier. Validation data generation algorithms are described in more detail below.

At step 9820, servers 9010 output the validation data. The validation data output may be output by servers 9010 in any one or more of a number of ways, for example any of the ways described above by which the servers 9010 output the identifier at step 9550.

The output validation data is sent to central controller 4230 of RPT device 4000 where it is received at step 9830. The validation data may be sent directly or indirectly to the central controller 4230 of RPT device 4000, for example via patient 1000 and/or local external device 4288 as described above for the identifier.

At step 9840 central controller 4230 generates a validation identifier. This step is performed by applying a validation identifier generation algorithm and using the validation data received at step 9830 as an input to that algorithm. The validation identifier generation algorithm reverses the effect of the validation data generation algorithm, i.e. when applied to validation data generated from an identifier using the validation identifier generation algorithm it produces the same identifier. Therefore, the validation identifier generated at step 9840 provides a mechanism for validating that the correct identifier is used to generate the combination of settings for configuring the RPT device 4000.

At step 9850, the central controller 4230 compares the validation identifier generated at step 9840 with the identifier received at step 9610. If the validation identifier matches the received identifier then this validates that the identifier has been correctly provided to central controller 4230, and the central controller 4230 proceeds to determine the combination of settings from the identifier at step 9620 and then configure the RPT device 4000 accordingly at step 9630. On the other hand, if the validation identifier does not match the received identifier then this indicates that an error has occurred, for example the patient 1000 may have entered the identifier incorrectly into a user input device, or the identifier may have been incorrectly communicated in some way. If this occurs then central controller initiates an error process at step 9860. The error process may comprise causing a display 4294 of the RPT device 4000 to display an error message to the patient 1000, or causing a message indicating an error to be sent to clinician 1500.

### 5.12.6.2 Validation Data - Second Embodiment

A method 9900 for validating an identifier received by the central controller 4230 of RPT device 4000 according to another form of the technology is illustrated in FIG. 14. Systems according to forms of the technology previously described are used to implement the method of FIG. 14.

In some forms of the technology the validation method 9900 shown in FIG. 14 forms part of the configuration method 9500 of FIG. 11. Therefore, FIG. 14 includes some of the same steps as are shown in FIG. 11. Where steps from FIG. 11 are omitted from FIG. 14 this is for ease of presentation in the figure and not because the steps do not occur in the validation method 9900.

Many of the steps of method 9900 are the same as those explained above in relation to method 9800. Where the methods differ is after the central controller 4230 has received the validation data at step 9830 and the identifier at step 9610.

In method 9900, the central controller 4230 generates further validation data from the received identifier at step 9910. Central controller 4230 may generate the further validation data by applying the same validation data generation algorithm as is applied by servers 9010 at step 9810. This provides a mechanism for validating that the correct identifier is used to generate the combination of settings for configuring the RPT device 4000.

At step 9920, the central controller 4230 compares the further validation data generated at step 9910 with the validation data received at step 9830. If the further validation data matches the received validation data then this validates that the identifier has been correctly provided to central controller 4230, and the central controller 4230 proceeds to determine the combination of settings from the identifier at step 9620 and then configure the RPT device 4000 accordingly at step 9630. On the other hand, if the further validation data does not match the received validation data then this indicates that an error has occurred, for example the patient 1000 may have entered the identifier incorrectly into a user input device, or the identifier may have been incorrectly communicated in some way. If this occurs then central controller initiates an error process at step 9860. The error process may comprise causing a display 4294 of the RPT device 4000 to display an error message to the patient 1000, or causing a message indicating an error to be sent to clinician 1500.

### 5.12.6.3 Validation Data

Validation data according to forms of the technology may be any data or element that has the capability of validating a combination of settings or identifier, for example upon the application of an appropriate algorithm or combination of algorithms.

Validation data may take any one or more of the forms described earlier in relation to the identifier. For example, validation data may be a character string. The validation may also be sent from one party or device to another party or device in a similar manner to that described above in relation to the identifier. Furthermore, in certain forms of the technology, the data representative of the validation data may be encrypted.

### 5.12.6.4 Validation Algorithms

As has been described, in forms of the technology, a validation data generation algorithm is used to generate validation data from an identifier, for example at steps 9810 and 9910. Furthermore, a validation identifier generation algorithm is used to generate a validation identifier from validation data in forms of the technology, for example at step 9840. The validation identifier generation algorithm reverses the effect of the validation data generation algorithm, i.e. when applied to validation data generated from an identifier using the validation identifier generation algorithm it produces the same identifier. That is, the validation identifier generation algorithm may be an operation that is inverse to the validation data generation algorithm.

In certain forms of the technology the validation identifier generation algorithm and the validation data generation algorithm are such that the validation data generated by the validation data generation algorithm is unique to each identifier input into that algorithm, and the validation identifier generated by the validation identifier generation algorithm is unique to each validation data input into that algorithm.

In forms of the technology the validation data generation algorithm may be a hash function, checksum function or fingerprint function. The validation identifier generation algorithm may be, where it exists, an inverse function to the validation data generation algorithm.

One advantage of method 9900 over method 9800 is that method 9900 does not use a validation identifier generation algorithm. Instead, the validation data generation algorithm is used twice: once at the ‘sender’ side by servers 9010 and once at the ‘receiver’ side by central controller 4230. Therefore method 9900 may use types of validation data generation algorithms that do not have an inverse, or where the inverse operation is computationally difficult.

### 5.12.6.5 Valid Identifier Validation

The examples of validation of the identifier given above enable validation of the identifier received by the central controller 4230 as being the identifier that corresponds to the combination of settings intended by the clinician 1500. In forms of the technology the validation process additionally or alternatively validates that the identifier is a valid identifier Iₓ.

In these forms of the technology the central controller 4230 performs a step of validating that the received identifier is a valid identifier after receiving the identifier at step 9610. This validating step may occur before, after or at the same time as other steps described occurring subsequent to step 9610 in methods 9500, 9800 and 9900 above.

In certain forms of the technology, the identifier comprises data and/or elements which are used to self-validate the identifier, and the validation step comprises applying an appropriate algorithm to the received identifier to confirm the validity of the received identifier using the self-validating data and/or elements. Examples of types of suitable self-validation data and/or elements, and/or self-validating algorithms include: checksum functions; check digits; and error-correcting codes. Specific examples include: Luhn algorithm; Verhoeff algorithm and Damm algorithm.

### 5.13 Verification of Rpt Device Settings

The preceding section describes methods and systems by which RPT device 4000 may be configured by selecting particular settings for any one or more of the operating parameters in accordance with which the central controller 4230 controls operation of the RPT device 4000. Forms of the technology also provide methods and/or systems by which the combination of settings with which the RPT device 4000 is configured can be verified. It may be useful for a clinician 1500, or other medical professional, to verify the configuration of the RPT device 4000 of a patient 1000 from time-to-time to ensure that the settings of the RPT device 4000 are still appropriate to the patient’s therapy needs. There may have been a change to the patient’s therapy needs that warrants a change in settings, or it may be useful to check that the RPT device settings have not unexpectedly changed, for example through a technical fault or the patient (or other person) inadvertently causing the settings to be changed.

### 5.13.1 Method of Verifying Configuration of RPT Device

FIG. 15 is a flow diagram of a method 8900 of verifying the configuration RPT device 4000 according to certain forms of the technology. FIG. 15 illustrates parties that may be involved in the implementation of method 8900, which in the form shown includes clinician 1500, servers 9010, patient 1000 or local external device 4288, and central controller 4230 of RPT device 4000.

At step 8910 the clinician 1500 requests that the settings of RPT device 4000 be verified. In one form of the technology, this step comprises the clinician 1500 asking the patient 1000 to initiate a settings verification process, for example asking the patient 1000 verbally or via an electronic message. In another form of the technology, the clinician 1500 may interact with a computing device 9040 to initiate a settings verification process, for example by clicking an appropriate button or icon on a graphical user interface. In this form, a message requesting verification of the settings of RPT device 4000 may be sent to patient 1000, local external device 4288 and/or another computing device 9040 associated with the patient 1000.

At step 8920 central controller is prompted to initiate a settings check. In one form of the technology the patient 1000 may manually initiate this by performing an action on a device. For example, the patient 1000 may interact with the RPT device 4000 to initiate a settings check. For example, the patient 1000 may push an “initiate settings check” button on an input device 4220 of the RPT device 4000, and may navigate an interface on a display 4294 of the RPT device 4000 to present such a button to the patient 1000. In another example, the patient 1000 may interact with a local external device 4288, for example a mobile computing device, in order to prompt the settings check. In this example, the mobile computing device may run an app that patient 1000 can interact with to select an “initiate settings check” button. In these forms of the technology, a message is sent to central controller 4230 indicating that a settings check is to be performed. In another example, the patient 1000 may play no part in initiating a settings check. For example, the computing device 9040 with which the clinician 1500 interacts may cause a communication to be sent to central controller 4230 to initiate a settings check without any communication being sent through a device of the patient 1000.

At step 8930 central controller 4230 queries the settings with which the RPT device 4000 is currently configured. In one form of the technology the combination of settings is stored in a memory 4260 of the RPT device 4000 and the central controller 4230 retrieves the settings from the memory 4260. At step 8940 central controller 4230 receives the combination of settings.

At step 8950 central controller 4230 determines an identifier from the received combination of settings. In certain forms, central controller 4230 uses any of the methods of determining an identifier from a combination of settings that have already been described with reference to the method 9500 for configuring RPT device 4000. The identifier determined by the central controller 4230 at step 8950 may be the same identifier, or a different identifier, to that which is determined by servers 9010 from the same combination of settings during configuration method 9500, i.e. the same or a different identifier-to-combination of settings correspondence (and therefore determination method) may be used.

At step 8960 the central controller outputs the identifier. The identifier may be output in any one or more of a number of ways. For example, the identifier may be displayed to patient 1000 on a display 4294 of RPT device 4000. Additionally, or alternatively, the identifier may be sent by data communication interface 4280 to a computing device 9040 associated with, for example, clinician 1500, or another healthcare professional, via network 9030. In one form of the technology, the identifier is first sent to patient 1000, for example by display on the local external device 4288 of the patient 1000, and at step 8970 the patient 1000 sends the identifier to another party, for example the clinician 1500. The identifier may be sent by any suitable communications protocol including email, SMS, FTP, HTTP, HTTPS. In other forms, the patient 1000 may send the identifier to the clinician 1500 via another means of communication, for example, orally or mail.

At step 8980 clinician 1500 and/or computing device 9040 (which may be associated with the clinician), as the case may be, receives the identifier.

At step 8990 the identifier is input to system 9000 and servers 9010. The manner in which this step occurs depends on the nature of the identifier. Examples for inputting an identifier to RPT device 4000 according to certain forms of the technology have been explained earlier. In certain forms of the technology, the identifier may be provided to servers 9010 in an analogous way to those described above in relation to RPT device 4000, that is, using similar input devices and mechanisms. Generally, the identifier is input into servers 9010 via one or more of network 9030 and computing device(s) 9040.

At step 8992 the servers 9010 of system 9000 receive the identifier. For example, data representative of the identifier may be sent from data communication network 9030 and/or computing device(s) 9040 to servers 9010.

At step 8994 the servers 9010 determine, from the identifier, the combination of settings for the RPT device 4000. The servers 9010 may determine the combination of settings from the identifier using any one or more of the methods described earlier by which the central controller RPT device 4000 may determine the combination of settings from the received identifier in configuration method 9500.

At step 8996 the servers 9010 output the combination of settings determined from the identifier at step 8994. In one form the combination of settings are output by displaying the settings on a display device of one or more computing device 9040. The combination of settings may display the setting and the corresponding operating parameter in association with one another, for example in a tabular format. In one form of the technology the servers 9010 output a subset of the settings determined from the identifier at step 8994. Alternatively, the servers 9010 may be configured to display only certain key settings that a clinician 1500 may be particularly interested in. A computing device 9040 displaying the key settings may present the clinician with the opportunity to view other settings if needed. An application run by servers 9010 may present the clinician with the opportunity to select which settings are displayed when running a settings verification process, and which are not displayed.

The clinician 1500 is able to review the output combination of settings and verify whether the RPT device 4000 is configured appropriately, for example by comparing the combination of settings to prescription data. In one form of the technology servers 9010 compare the combination of settings determined at step 8994 with data on the intended combination of settings for the patient 1000 in the medical records of the patient 1000 (which may be stored in memory 9014) and outputs an indication as to how they compare, for example highlighting any discrepancies for easy identification by the clinician 1500 or outputting a confirmation of a complete match between the intended and actual combination of settings. The clinician 1500 can relay the outcome of the settings verification process to the patient 1000 and, if necessary, the RPT device 4000 can be re-configured appropriately, for example using a configuration method according to a form of the technology explained above.

### 5.14 Glossary

For the purposes of the present technology disclosure, in certain forms of the present technology, one or more of the following definitions may apply. In other forms of the present technology, alternative definitions may apply.

### 5.14.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient’s respiratory system.

Flow therapy: Respiratory therapy comprising the delivery of a flow of air to an entrance to the airways at a controlled flow rate referred to as the treatment flow rate that is typically positive throughout the patient’s breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient’s face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal = 100 Pa = 100 N/m² = 1 millibar ~ 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 5.14.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient’s respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea. According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device’s estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient’s respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.14.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator’s inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ) = 0 when Φ = 1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS = IPAP - EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (SIT): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient’s efforts.

### 5.15 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

5.16 REFERENCE SIGNS LIST

| | |
|---|---|
| 1000 | Patient |
| 1100 | Bed partner |
| 1500 | Clinician |
| 2000 | Headbox |
| 2015 | EOG electrode |
| 2020 | EEG electrode |
| 2025 | ECG electrode |
| 2030 | Submental EMG electrode |
| 2035 | Snore sensor |
| 2040 | Respiratory inductance plethysmogram (respiratory effort sensor) |
| 2045 | Respiratory inductance plethysmogram (respiratory effort sensor) |
| 2050 | Oro-nasal cannula |
| 2055 | Photoplethysmograph (pulse oximeter) |
| 2060 | Body position sensor |
| 3000 | Patient interface |
| 3100 | Seal-forming structure |
| 3200 | Plenum chamber |
| 3300 | Positioning and stabilising structure |
| 3400 | Vent |
| 3600 | Connection port |
| 3700 | Forehead support |
| 3800 | Nasal cannula |
| 3810 | Nasal prongs |
| 3820 | Air supply lumens |
| 4000 | RPT device |
| 4010 | External housing |
| 4012 | Upper portion |
| 4014 | Lower portion |
| 4015 | Panel(s) |
| 4016 | Chassis |
| 4018 | Handle |
| 4020 | Pneumatic block |
| 4110 | Air filter |
| 4112 | Inlet air filter |
| 4120 | Muffler |
| 4122 | Inlet muffler |
| 4124 | Outlet muffler |
| 4140 | Pressure generator |
| 4142 | Blower |
| 4144 | Brushless DC motor |
| 4170 | Air circuit |
| 4180 | Supplementary gas |
| 4200 | Electrical components |
| 4202 | Printed Circuit Board Assembly (PCBA) |
| 4210 | Electrical power supply |
| 4220 | Input device |
| 4230 | Central controller |
| 4240 | Therapy device controller |
| 4250 | Protection circuit |
| 4260 | Memory |
| 4270 | Transducers |
| 4272 | Pressure sensor |
| 4274 | Flow rate sensor |
| 4276 | Motor speed transducer |
| 4280 | Data communication interface |
| 4282 | Remote external communication network |
| 4284 | Local external communication network |
| 4286 | Remote external device |
| 4288 | Local external device |
| 4290 | Output device |
| 4300 | Algorithms |
| 4310 | Pre-processing module |
| 4312 | Interface pressure estimation |
| 4314 | Vent flow rate estimation |
| 4316 | Leak flow rate estimation |
| 4318 | Respiratory flow rate estimation |
| 4320 | Therapy engine module |
| 4321 | Phase determination |
| 4322 | Waveform determination |
| 4323 | Ventilation determination |
| 4324 | Inspiratory flow limitation determination |
| 4325 | Apnea / hypopnea determination |
| 4326 | Snore determination |
| 4327 | Airway patency determination |
| 4328 | Target ventilation determination |
| 4329 | Therapy parameter determination |
| 4330 | Therapy control module |
| 4340 | Method / algorithm |
| 4170 | Air circuit |
| 4171 | Heated air circuit |
| 5000 | Humidifier |
| 5002 | Humidifier inlet |
| 5004 | Humidifier outlet |
| 5006 | Humidifier base |
| 5110 | Humidifier reservoir |
| 5130 | Humidifier reservoir dock |
| 5210 | Transducer |
| 5212 | Air pressure sensor |
| 5214 | Air flow rate transducer |
| 5216 | Temperature sensor |
| 5218 | Humidity sensor |
| 5240 | Heating element |
| 5250 | Humidifier controller |
| 5251 | Central humidifier controller |
| 5252 | Heating element controller |
| 5254 | Heated air circuit controller |
| 7100 | Monitoring apparatus |
| 7200 | Screening / diagnosis / monitoring device |
| 7210 | Processor |
| 7220 | Communication interface |
| 7230 | Non-transitory computer readable memory / storage medium |
| 7240 | Data |
| 7250 | Processor control instructions (code) |
| 7260 | Data input interface |
| 8000 | Oxygen concentrator |
| 8002 | Air inlet |
| 8004 | Inlet muffler |
| 8006 | Accumulator |
| 8008 | Outlet |
| 8020 | Inlet valve |
| 8022 | Inlet valve |
| 8030 | Outlet valve |
| 8032 | Outlet valve |
| 8040 | Check valve |
| 8042 | Check valve |
| 8050 | Flow restrictor |
| 8052 | Flow restrictor |
| 8054 | Flow restrictor |
| 8056 | Valve |
| 8058 | Valve |
| 8100 | Canister |
| 8102 | First canister |
| 8104 | Second canister |
| 8200 | Compression system |
| 8300 | Controller |
| 8210 | Processor |
| 8320 | Memory |
| 8500 | Outer housing |
| 8502 | Compression system inlet |
| 8504 | Cooling system passive inlet |
| 8506 | Port |
| 8600 | Control panel |
| 8602 | Charging input port |
| 8900 | Method |
| 8910-8996 | Method steps |
| 9000 | System |
| 9010 | Servers |
| 9012 | Processor |
| 9014 | Memory |
| 9016 | Instructions |
| 9018 | Data |
| 9030 | Communication network |
| 9032 | Wired communication network |
| 9034 | Wireless communication network |
| 9040 | Computing device |
| 9042 | Desktop or laptop computer |
| 9044 | Smartphone |
| 9046 | Tablet |
| 9100 | General architecture |
| 9110 | Processor |
| 9120 | Memory/data storage |
| 9122 | Control instructions |
| 9124 | Stored data |
| 9130 | Input/output (I/O) device |
| 9132 | Display |
| 9134 | Speaker |
| 9136 | Haptic feedback device |
| 9138 | Physical input device |
| 9140 | Optical sensor |
| 9142 | Inertial sensor |
| 9150 | Communication interface |
| 9160 | Communication interface |
| 9500 | Method |
| 9510-9630 | Method steps |
| 9700 | Data array |
| 9800 | Method |
| 9810-9860 | Method steps |
| 9900 | Method |
| 9910-9920 | Method steps |

1. A processor-implemented method of configuring a respiratory device, the respiratory device comprising a processor configured to control operation of the respiratory device in accordance with a plurality of operating parameters, each of the plurality of operating parameters being able to be set to a plurality of settings, the method comprising:
receiving an identifier, wherein the identifier is one identifier out of a plurality of identifiers, wherein each of the plurality of identifiers corresponds to a combination of settings out of a plurality of combinations of settings, wherein each of the combinations of settings comprises a setting out of the plurality of settings for each of the plurality of operating parameters;
validating the identifier as corresponding to an intended combination of settings, the step of validating comprising:
receiving first validation data, the first validation data generated from the identifier using a validation data generation algorithm; and
generating second validation data from the identifier using the validation data generation algorithm; and
comparing the first validation data with the second validation data;
wherein, if the second validation data matches the first validation data, the method further comprises:
determining, from the identifier, the combination of settings corresponding to the identifier; and
configuring the respiratory device to operate in accordance with the determined combination of settings.

2. A processor-implemented method as claimed in claim 1, wherein the combination of settings corresponding to each of the plurality of identifiers is unique to each identifier.

3. A processor-implemented method as claimed in claim 1, wherein the identifier is received as data representative of a character string.

4. A processor-implemented method as claimed in claim 1, wherein the method comprises receiving the identifier from a user input device.

5. A processor-implemented method as claimed in claim 4, wherein the user input device is a mobile computing device configured to send the identifier directly or indirectly to the processor.

6. A processor-implemented method as claimed in claim 4, wherein the step of receiving the identifier comprises receiving optical data representative of an optically machine-readable code, and generating the identifier from the optical data.

7. A processor-implemented method as claimed in claim 4, wherein the step of receiving the identifier comprises receiving acoustic data representative of a plurality acoustic tones, and generating the identifier from the acoustic data.

8. A processor-implemented method as claimed in claim 4, wherein the user input device is a keypad on the respiratory device.

9. A processor-implemented method as claimed in claim 1, wherein the step of determining the combination of settings from the identifier comprises identifying the combination of settings corresponding to the identifier in a data array, the data array storing each of the plurality of identifiers and the corresponding combination of settings out of the plurality of combination of settings in relation to each other.

10-14. (canceled)

15. A processor-implemented method as claimed in claim 1, wherein the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

16-25. (canceled)

26. A processor-implemented method of generating an identifier for use in verifying the configuration of a respiratory device, the respiratory device comprising a processor configured to control operation of the respiratory device in accordance with a plurality of operating parameters, each of the plurality of operating parameters being able to be set to a plurality of settings, the method comprising:
receiving a present combination of settings of the respiratory device, wherein the present combination of settings is one combination of settings out of a plurality of combinations of settings for the respiratory device, and wherein each of the combinations of settings comprises a setting out of the plurality of settings for each of the plurality of operating parameters of the respiratory device;
determining, from the received combination of settings, an identifier, wherein the identifier is one identifier out of a plurality of identifiers, wherein each of the plurality of identifiers corresponds to one combination of settings out of the plurality of combinations of settings; and
outputting the identifier from the respiratory device.

27. A processor-implemented method as claimed in claim 26, wherein the combination of settings corresponding to each of the plurality of identifiers is unique to each identifier.

28. A processor-implemented method as claimed in claim 26, wherein the identifier is output as data representative of a character string.

29. A processor-implemented method as claimed in claim 26, wherein the identifier is output to a display of the respiratory device.

30. A processor-implemented method as claimed in claim 26, wherein the identifier is output as optical data representative of an optically machine-readable code.

31. A processor-implemented method as claimed in claim 26, wherein the identifier is output as acoustic data representative of a plurality acoustic tones.

32. A processor-implemented method as claimed in claim 26, wherein the step of determining the identifier from the received combination of settings comprises identifying the identifier corresponding to the received combination of settings in a data array, the data array storing each of the plurality of identifiers and the corresponding combination of settings out of the plurality of combination of settings in relation to each other.

33. A processor-implemented method as claimed in claim 26, wherein the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

34. A processor-implemented method of verifying the configuration of a respiratory device by a processor remote from the respiratory device, wherein the respiratory device is configured to operate in accordance with a plurality of operating parameters, each of the plurality of operating parameters being able to be set to a plurality of settings, the method comprising:
receiving an identifier output from the respiratory device, wherein the identifier is one identifier out of a plurality of identifiers, wherein each of the plurality of identifiers corresponds to a present combination of settings out of a plurality of combinations of settings, wherein each of the combinations of settings comprises a setting out of the plurality of settings for each of the plurality of operating parameters;
determining, from the identifier, the present combination of settings corresponding to the identifier; and
outputting the present combination of settings.

35. A processor-implemented method as claimed in claim 34, wherein the combination of settings corresponding to each of the plurality of identifiers is unique to each identifier.

36. A processor-implemented method as claimed in claim 34, wherein the identifier is received as data representative of a character string.

37. A processor-implemented method as claimed in claim 34, wherein the method comprises receiving the identifier from a user input device.

38. A processor-implemented method as claimed in claim 37, wherein the user input device is a mobile computing device configured to send the identifier directly or indirectly to the processor.

39. A processor-implemented method as claimed in claim 34, wherein the step of receiving the identifier comprises receiving optical data representative of an optically machine-readable code, and generating the identifier from the optical data.

40. A processor-implemented method as claimed in claim 34, wherein the step of receiving the identifier comprises receiving acoustic data representative of a plurality acoustic tones, and generating the identifier from the acoustic data.

41. A processor-implemented method as claimed in claim 34, wherein the step of determining the present combination of settings from the identifier comprises identifying the combination of settings corresponding to the identifier in a data array, the data array storing each of the plurality of identifiers and the corresponding combination of settings out of the plurality of combination of settings in relation to each other.

42. A processor-implemented method as claimed in claim 34, wherein the plurality of operating parameters comprise parameters relating to any one or more of: a patient receiving respiratory therapy; the respiratory device; a peripheral device used in conjunction with the respiratory device; delivery of respiratory therapy; breathable gas delivered by the respiratory device; one or more alarm conditions; and humidification.

43. A respiratory device comprising:
a flow generator for generating a flow of air for delivery to a patient’s airways; and
a processor configured to perform the processor-implemented method of claim 1.

44. A system comprising a processor configured to perform the processor-implemented method of claim 34.
